(19) United States
(12) Patent Application Publication
(10) Pub. No.: US 2025/0230256 A1
(43) Pub. Date: Jul. 17, 2025

(54) COMBINATION THERAPY OF BISPECIFIC ANTIBODIES AGAINST CEACAM5 AND CD47 AND BISPECIFIC ANTIBODIES AGAINST CEACAM5 AND CD3

(71) Applicant: LAMKAP BIO BETA LTD, Pfaeffikon (CH)

(72) Inventors: Vanessa BUATOIS, Contamine-Sarzin (FR); Anja SECKINGER, Pfaeffikon (CH); Dirk HOSE, Pfaeffikon (CH)

(21) Appl. No.: 18/875,477

(22) PCT Filed: Jun. 15, 2023
(86) PCT No.: PCT/EP2023/066141

§ 371 Date: Dec. 16, 2024

(30) Foreign Application Priority Data
Jun. 16, 2022 (EP) 22179484

(51) Int. Cl. C07K 16/30; A61K 39/00; A61P 35/00; C07K 16/28

(52) U.S. Cl. CPC C07K 16/3007; A61P 35/00; C07K 16/2803; C07K 16/2809; A61K 2039/507; A61K 2039/545; C07K 2317/31; C07K 2317/565; C07K 2317/76; C07K 2317/92

(57) ABSTRACT

The present invention relates to a bispecific antibody which bind to human carcinoembryonic antigen CEACAM5 and to human CD47 for use in the treatment of cancer with a bispecific antibody which bind to human carcinoembryonic antigen CEACAM5 and to human CD3ε, such combination and their use in the treatment of diseases.

### REFERENCE TO SEQUENCE LISTING

The content of the electronically submitted sequence listing filed with the application is incorporated herein by reference in its entirety.

### FIELD OF THE INVENTION

The present invention relates to a bispecific antibody which bind to human carcinoembryonic antigen five (CEACAM5, CEA) and to human CD47, as described below, (CEA×CD47 bispecific antibody) for use in the treatment of cancer combined with a bispecific antibody which bind to CEA and to human CD38, as described below, (CEA×CD3 bispecific antibody), such combinations, and their use in the treatment of diseases.

### BACKGROUND OF THE INVENTION

CEA belongs to the family of CEA-related cell adhesion molecules (CEACAMs) that comprises 12 closely related proteins in humans encoded by 22 genes divided among the CEACAM and pregnancy-specific glycoproteins (PSG) subgroups on chromosome 19q13 (Beauchemin N & Arabzadeh A, Cancer Metastasis Rev. 2013). CEACAMs are involved in a variety of physiological processes such as cell-cell recognition and modulate cellular processes ranging from the shaping of tissue architecture and neovascularization to the regulation of insulin homeostasis, and T-cell proliferation: CEACAMs have also been identified as receptors for host-specific viruses and bacteria (Kuespert K et al., Curr Opin Cell Biol. 2006). CEA (CEACAM5 or CD66e: UniProtKB-P06731) is present early in embryonic and fetal development and maintains its expression in normal adult tissues. Its main site of expression is in columnar epithelial and goblet cells of the colon, particularly in the upper third of the crypt and at the free luminal surface.

CEA is (over-)expressed in tumors of epithelial origin, including but not limited to colorectal, gastric, lung, and pancreatic carcinomas (reviewed in Beauchemin N & Arabzadeh A, Cancer Metastasis Rev. 2013). It loses its apical expression resulting in distribution over the entire cell surface (Hammarstrom, Semin Cancer Biol 1999). Given the overexpression often observed in CEA positive tumors and the distribution of tumor cell based CEA over the entire surface, CEA is an interesting target for immunotherapeutical attack of cancer cells while sparing normal cells of the same tissue.

A method for treating CEA expressing cancer by a combination of a human PD-1 axis antagonist and a T-cell redirecting and activating anti-CEA/anti-CD3 bispecific antibody (Cibisatamab) is mentioned in US20140242079 and WO2017118657 (each of which is incorporated by reference in its entirety) and clinical results have been presented at the ASCO annual meeting 2017 (Tabernero et al., J Clin Oncol 35, 2017 (suppl: abstr 3002)). The only active recruiting clinical trial in June 2022 with Cibisatamab is a trial in combination with a FAP-4-1BBL bispecific antibody which can induce additional activation of T-cells via stimulation of the costimulatory receptor 4-1BB on T-cells (ClinicalTrial.gov Identifier NCT04826003). Patients are pre-treated with the B-cell depleting agent Obinutuzumab to avoid formation of anti-drug antibodies (ADA) and consecutive loss of exposure. ADA formation and loss of exposure of Cibisatamab have been observed in the clinical trial of cibisatamab mentioned above, in monotherapy as well as in combination with a PD-L1 inhibitor).

A method of treating tumors by administering immune checkpoint antagonists binding two or more different targets of an immune checkpoint pathway, and a T-cell redirecting agent binding to CEA and a T-cell surface antigen is mentioned in WO2015112534. A class I antibody binding to CEACAM5 and granulocytes is mentioned in US20110064653.

Human CD47 (UniProtKB—Q08722 (CD47 HUMAN; IAP)) is a transmembrane protein that binds the ligands thrombospondin-1 (TSP-1) and signal-regulatory protein alpha (SIRPα; CD172a; UniProtKB P78324) and can act as a “don't eat me” signal to the immune system, especially for macrophages which express SIRPα. Potent inhibition (low IC50) of the binding of SIRPα to CD47 on the surface of tumor cells is a measure to increase the phagocytosis of tumor cells by macrophages. CD47 is involved in a range of cellular processes, including apoptosis, proliferation, adhesion, and migration. Furthermore, it plays a key role in immune and angiogenic responses. CD47 is overexpressed in tumor cells from patients with both hematological and solid tumors. Antibodies against CD47 are described in the state of the art and have shown promising preclinical and early clinical activity in different tumor entities, including hematological malignancies such as lymphoma and solid tumors, for example gastric cancer (Weiskopf K., European Journal of Cancer 76 (2017) 100-109; Huang Y et al., J Thorac Dis 2017; 9 (2):E168-E174; Kaur et al., Antibody Therapeutics, 3 (2020) 179-192). Antibodies of the IgG1 subclass that bind CD47 can result in the depletion of platelets (thrombocytopenia) and reduction in the number of red blood cells (RBC, anemia) in a Fc-dependent manner (see e.g. US20140140989). For avoiding this adverse effect, in WO2017196793 there is described a mutant form of the IgG4 subclass of an anti-CD47 antibody (IgG4PE, with the S228P mutation as well as a L235E mutation to reduce FcγR binding). Such an anti-CD47 antibody with severely reduced FcγR binding and effector function does not result in such platelet depletion. A single domain bispecific antibody against CD47 and CD20 was described by von Bommel P E et al. (Oncoimmunol. 7 (2018) e386361) and Piccione E C et al. (mAbs 7 (2015)946-956). Dheilly E. et al. (Mol. Thera. 25 (2017) 523-533; see also WO2014087248) describe a bispecific antibody against CD19 and CD47.

Bispecific antibodies against CEACAM5 and CD47 comprising a common heavy chain of SEQ ID NO:5 (VH-CH1) and a CD47-interacting variable light chain region VL of SEQ ID NO:10 are described in WO2019234576, EP19213002, and U.S. 62/943,726 (incorporated by reference in their entirety). A bispecific antibody against CD19 and CD47 comprising a common heavy chain of SEQ ID NO:5 and a CD47-interacting variable light chain region VL of SEQ ID NO:10 is described in WO2014087248 (incorporated by reference in its entirety). WO2018098384 relates to a bispecific antibody co-targeting CD47 and CEACAM5. EP3623388 relates to bispecific binding molecules comprising a tumor-targeting arm and a fusion protein with low affinity for blocking the interaction between CD47 and SIRPα. WO 2018/057955 relates to bispecific antibodies binding both CD47 and mesothelin and comprising a common heavy chain. WO2019016411 relates to bispecific antibody molecules targeting CD47 and a tumor antigen.

Despite certain progress in the treatment of locally advanced or especially metastatic solid cancer types, new anti-cancer drug induced significant increase of progression-free (PFS) and/or overall survival (OS) of patients suffering from advanced cancer like colorectal cancer, pancreatic cancer, lung cancer etc. is still rather limited and there is usually no cure. Much hope has been put into cancer immunotherapy and there is certain, but limited, successes. Tumors develop measures to protect from destruction by T-effector cells and other immune cells like macrophages. Cancer immunotherapy-based strategies in the last decade(s) have had some success in counteracting these tumor protective measures and re-directing T cells against cancer cells. The most prominent examples of such strategies are inhibitors/activators of certain immune checkpoints. For example, checkpoint inhibitors like PD-1 axis antagonists have shown to re-activate T-effector cells to fight certain solid cancers. But not all solid tumor types are responsive to PD-1 axis antagonists, and, even in those responsive types, often much less than 50% of patients have a relevant benefit from e.g. treatment with an anti-PD-1 or PD-L1 antibody. For example, less than 10% of the patients with advanced colorectal cancer are eligible to therapy with inhibitors of the PD-1 axis (especially the approximately 4% of advanced colorectal cancer patients showing Microsatellite Instability MSI in their cancer have some benefit).

Adoptive T-cell therapy with chimeric antigen receptor (CAR) T-cells and therapy with T-cell bispecific antibodies delivered promising clinical results in hematological malignancies. But clinical studies with adoptive T-cell therapies, e.g. CAR T-cells, in various solid tumors mostly showed no or only minor response rates (e.g. Xu et al., Expert Review of Anticancer Therapy 2017, 17, 1099-1106; Greenbaum et al., Biol Blood Marrow Transplant 2020 October; 26(10):1759-1769).

US20140242079, WO2017055389, US20140242080, WO2007071426, WO2013012414, WO2015112534, WO2017118675, US20140242079, and Bacac et al. (Clin. Cancer Res., 22(13), 3286-97 (2016)) describe CEA×CD3 T-cell bispecific antibodies.

T-cell bispecific antibodies TAA×CD3 (TAA=Tumor associated antigen like CEA and many others) are highly efficient in patients with hematological malignancies like Multiple Myeloma, B-cell malignancies like e.g. diffuse large B-cell lymphoma, follicular lymphoma etc. Clinical results with Cibisatamab CEA×CD3 show that there is efficacy of TAA×CD3 also in advanced solid tumors (see text above) but much less than achieved with e.g. CD20×CD3 or BCMA×CD3 etc in hematological malignancies (Moreau P1., N Engl J Med., 2022 Jun. 5. doi: 10.1056/NEJMoa2203478). Adding PD-1 axis inhibitors may add efficacy, but if at all only limited. Adding a bispecific antibody or fusion protein agonistic at a costimulatory T-cell receptor like CD28 or 4-1BB increases efficacy in preclinical tests, but also toxicity, e.g. increased cytokine release. Instead of aiming for additional activation of T-cells, it could be more successful to add a therapeutic agent re-directing to the tumor cells other immune cells, especially macrophages. This invention deals with bispecific antibodies CEA×CD47 re-directing and activating macrophages against CEACAM5-expressing solid tumors in combination therapy especially with CEA×CD3 T-cell bispecific antibodies to increase the tumor cell killing effect of the CEA×CD3 bispecific antibodies and to avoid, in contrast to the combination with bispecific agonists at T-cell co-stimulatory receptors, increased risk of Cytokine Release Syndrome CRS and potential T-cell exhaustion.

Bispecific antibodies against CEACAM5 and CD47 are described in WO2019234576 and WO2021110647. One bispecific antibody described in WO2019234576 is K2AC22 (SEQ ID NO:65 of WO2019234576 shows the light chain of the CEACAM5 binding part of K2AC22, SEQ ID NO:5 of WO2019234576 shows the common heavy chain of K2AC22 and SEQ ID NO:11 shows the light chain of the CD47 binding part of K2AC22). In WO2019234576, for K2AC22, increased tumor cell killing in combination with a CEA×CD3 called CEA-TCB has been shown. To the best of our knowledge the CEA-TCB is cibisatamab or at least a CEA×CD3 with the same basic structure as cibisatamab (2+1 format) binding to the same CEA epitope as cibisatamab. An object of the present invention is to provide a method for treating or delaying progression of cancer in an individual comprising administering to the individual an effective amount of a bispecific antibody against CEACAM5 and CD47 in combination with bispecific antibodies against CEACAM5 and CD3. An object of the invention is an advantageous combination of a CEA×CD47 bispecific antibody and a CEA×CD3 bispecific antibody. Such a combination is further described in the present invention.

The bispecific antibody against CEACAM5 and CD3 as used in the combination and method according to the invention is described in WO2021053587. The bispecific antibody against CEACAM5 and CD47 as used in the combination and method according to the invention is described in PCT/IB2021/061983 (WO2022130348).

### SUMMARY OF THE INVENTION

In one aspect, the present invention provides a bispecific antibody against CEACAM5 and CD47 as described below (further named also as CEA×CD47 bispecific antibody) for use in the treatment of cancer in combination with a bispecific antibody against CEACAM5 and CD3ε as described below (CEA×CD3 bispecific antibody).

In one aspect, the present invention provides a method for treating or delaying progression of cancer in an individual comprising administering to the individual an effective amount of a bispecific antibody against CEACAM5 and CD47 as described below (further named also as CEA×CD47 bispecific antibody) in combination with a bispecific antibody against CEACAM5 and CD3ε as described below (CEA×CD3 bispecific antibody). In one aspect, the present invention provides such a combination. The CEA×CD47 bispecific antibodies as used in the method or combination according to the invention induce high phagocytic activity against tumor cells, both against tumor cells expressing CEACAM5 in high amounts and against tumor cells expressing CEACAM5 in low amounts. In one embodiment, the CEA×CD47 bispecific antibodies induce their anti-tumor effects mainly via optimized phagocytosis/antibody-dependent cellular phagocytosis (ADCP) due to involvement of immune cells, especially macrophages. In one embodiment, the CEA×CD47 bispecific antibodies as used according to the invention show a decreased ratio of CEACAM3 to CEACAM5 binding affinity respectively increased ratio of KD relative to the CEACAM5×CD47 antibody K2AC22. In one embodiment, the CEA×CD47 bispecific antibody as used according to the invention inhibit the binding of SIRPα to CD47 expressed on tumor cells and increase phagocytosis of tumor cells. In one embodiment, the CEACAM5×CD3 bispecific antibody as used according to the invention is a kappa lambda bispecific antibody that fully retain the sequence and architecture of human IgG antibodies and therefore low risk of immunogenicity causing ADA formation and potential loss of exposure.

The combination according to the invention is also suitable for use in the treatment of tumors, especially in the treatment of solid tumors.

In one aspect, the present invention provides
A) a first bispecific antibody comprising a first binding part specifically binding to human CEACAM5 (further named also as “CEA”) and a second binding part specifically binding to human CD47 (further named also as “CD47”) characterized in that:
a) the first binding part comprises a heavy chain variable region comprising a CDRH1 of SEQ ID NO:23, a CDRH2 of SEQ ID NO:24 and a CDRH3 of SEQ ID NO:25, and a light chain variable region comprising a CDRL1 of SEQ ID NO:35, CDRL2 of SEQ ID NO:36, and CDRL3 of SEQ ID NO:37,
b) the second binding part comprises a heavy chain variable region comprising a CDRH1 of SEQ ID NO:23, a CDRH2 of SEQ ID NO:24, and a CDRH3 of SEQ ID NO:25, and as light chain variable region a light chain variable region comprising a CDRL1 of SEQ ID NO:29, a CDRL2 of SEQ ID NO:30, and a CDRL3 of SEQ ID NO:31, for use in the treatment of cancer in combination with
B) a second bispecific antibody comprising a first binding part specifically binding to human CEACAM5 and a second binding part specifically binding to human CD3ε (further named also as “CD3”) characterized in that:
a) the first binding part comprises a heavy chain variable region comprising a CDRH1 of SEQ ID NO:2, a CDRH2 of SEQ ID NO:3 and a CDRH3 of SEQ ID NO:4, and a light chain variable region comprising
a CDRL1 of SEQ ID NO:18, CDRL2 of SEQ ID NO:19, and CDRL3 of SEQ ID NO:20, and a heavy chain variable region comprising a CDRH1 of SEQ ID NO:2, a CDRH2 of SEQ ID NO:3 and a CDRH3 of SEQ ID NO:4, and a light chain variable region comprising a CDRL1 of SEQ ID NO:6, a CDRL2 of SEQ ID NO:7, and a CDRL3 of SEQ ID NO:8.

In one aspect, the invention comprises such method of treatment.

In one aspect, the present invention provides
A) a first bispecific antibody comprising a first binding part specifically binding to human CEACAM5 and a second binding part specifically binding to human CD47 characterized in that:
a) the first binding part comprises a heavy chain variable region of SEQ ID NO:26 and a light chain variable region of SEQ ID NO:38, and
b) the second binding part comprises a heavy chain variable region of SEQ ID NO:26, and a light chain variable region of SEQ ID NO:32, for use in the treatment of cancer in combination with
B) a second bispecific antibody comprising a first binding part specifically binding to human CEACAM5 and a second binding part specifically binding to human CD3ε characterized in that:
a) the first binding part comprises a heavy chain variable region of SEQ ID NO:1 and a light chain variable region of SEQ ID NO:5, and
b) the second binding part comprises a heavy chain variable region of SEQ ID NO:1, and a light chain variable region of SEQ ID NO:21.

In one aspect, the invention comprises such method of treatment.

In one aspect, the present invention provides
A) a first bispecific antibody comprising a first binding part specifically binding to human CEACAM5 and a second binding part specifically binding to human CD47 characterized in that:
a) the first binding part comprises a heavy chain region of SEQ ID NO:27 and a light chain of SEQ ID NO: 39, and
b) the second binding part comprises a heavy chain region of SEQ ID NO:27, and a light chain of SEQ ID NO:33, for use in the treatment of cancer in combination with
B) a second bispecific antibody comprising a first binding part specifically binding to human CEACAM5 and a second binding part specifically binding to human CD3c characterized in that:
a) the first binding part comprises a heavy chain region of SEQ ID NO: 10 and a light chain of SEQ ID NO:22, and
b) the second binding part comprises a heavy chain region of SEQ ID NO:10, and a light chain of SEQ ID NO:9.

In one aspect, the invention comprises such method of treatment.

In one aspect, the present invention provides a combination of
A) a first bispecific antibody comprising a first binding part specifically binding to human CEACAM5 and a second binding part specifically binding to human CD47, characterized in that:
A) a first bispecific antibody comprising a first binding part specifically binding to human CEACAM5 (further named also as “CEA”) and a second binding part specifically binding to human CD47 (further named also as “CD47”) characterized in that:
a) the first binding part comprises a heavy chain variable region comprising a CDRH1 of SEQ ID NO:23, a CDRH2 of SEQ ID NO:24 and a CDRH3 of SEQ ID NO:25, and a light chain variable region comprising a CDRL1 of SEQ ID NO:35, CDRL2 of SEQ ID NO:36, and CDRL3 of SEQ ID NO:37,
b) the second binding part comprises a heavy chain variable region comprising a CDRH1 of SEQ ID NO:23, a CDRH2 of SEQ ID NO:24, and a CDRH3 of SEQ ID NO:25, and as light chain variable region a light chain variable region comprising a CDRL1 of SEQ ID NO:29, a CDRL2 of SEQ ID NO:30, and a CDRL3 of SEQ ID NO:31, and
B) a second bispecific antibody comprising a first binding part specifically binding to human CEACAM5 and a second binding part specifically binding to human CD3ε (further named also as “CD3”) characterized in that:
a) the first binding part comprises a heavy chain variable region comprising a CDRH1 of SEQ ID NO:2, a CDRH2 of SEQ ID NO:3 and a CDRH3 of SEQ ID NO:4, and a light chain variable region comprising
a CDRL1 of SEQ ID NO:18, CDRL2 of SEQ ID NO:19, and CDRL3 of SEQ ID NO:20, and a heavy chain variable region comprising a CDRH1 of SEQ ID NO:2, a CDRH2 of SEQ ID NO:3 and
a CDRH3 of SEQ ID NO:4, and a light chain variable region comprising a CDRL1 of SEQ ID NO:6, a CDRL2 of SEQ ID NO:7, and a CDRL3 of SEQ ID NO:8.

In one aspect, the present invention provides a combination of
A) a first bispecific antibody comprising a first binding part specifically binding to human CEACAM5 and a second binding part specifically binding to human CD47 characterized in that:
a) the first binding part comprises a heavy chain variable region of SEQ ID NO:26 and a light chain variable region of SEQ ID NO:38, and
b) the second binding part comprises a heavy chain variable region of SEQ ID NO:26, and a light chain variable region of SEQ ID NO:32, and
B) a second bispecific antibody comprising a first binding part specifically binding to human CEACAM5 and a second binding part specifically binding to human CD3ε characterized in that:
a) the first binding part comprises a heavy chain variable region of SEQ ID NO:1 and a light chain variable region of SEQ ID NO:5, and
b) the second binding part comprises a heavy chain variable region of SEQ ID NO: 1, and a light chain variable region of SEQ ID NO:21.

In one aspect, the present invention provides a combination of
A) a first bispecific antibody comprising a first binding part specifically binding to human CEACAM5 and a second binding part specifically binding to human CD47 characterized in that:
a) the first binding part comprises a heavy chain region of SEQ ID NO:27 and a light chain of SEQ ID NO: 39, and
b) the second binding part comprises a heavy chain region of SEQ ID NO:27, and a light chain of SEQ ID NO:33, and
B) a second bispecific antibody comprising a first binding part specifically binding to human CEACAM5 and a second binding part specifically binding to human CD3ε characterized in that:
a) the first binding part comprises a heavy chain region of SEQ ID NO: 15 and a light chain of SEQ ID NO:22, and
b) the second binding part comprises a heavy chain region of SEQ ID NO:15, and a light chain of SEQ ID NO:9.

The invention comprises further embodiments of this aspect:

In one embodiment, the constant and variable framework region sequences of both antibodies are human.

In one embodiment, both antibodies are characterized in that each of the first and second binding part comprises an immunoglobulin heavy chain and an immunoglobulin light chain. In one embodiment, both antibodies are full-length antibodies. In one embodiment, the bispecific antibodies are characterized in being of human IgG1 type.

In one embodiment, both antibodies are characterized in being monovalent for the first binding part and monovalent for the second binding part.

In another embodiment, the first antibody as used according to the invention is characterized in being glycoengineered to have an Fc region with modified oligosaccharides. In another embodiment, the first bispecific antibody according to the invention is characterized in comprising a Fc region that has been glycoengineered to have a reduced number of fucose residues as compared to the same bispecific antibody that has not been glycoengineered.

In one embodiment, the first antibody as used according to the invention is characterized by a ratio of the KD values for the binding to recombinant CEACAM3 and recombinant CEACAM5 of a factor of 100 or more (Example 3, Table 2).

In one embodiment, the first antibody as used according to the invention is characterized by a ratio of the KD values for the binding to recombinant CEACAM3 and recombinant CEACAM5 of a factor of between 100 and 200.

In one embodiment, the first antibody as used in this invention has a relative uncoupling of binding to CEACAM5 and CEACAM3 (discriminative binding). Despite an, in comparison to bispecific CEA×CD47 antibody K2AC22, increased binding to the full length recombinant human CEACAM5 protein, the binding to full length recombinant human CEACAM3 does not increase proportionally.

The quotient/ratio of the KD for the binding to the full length CEACAM3 vs. CEACAM5 shows an increase from 83 (K2AC22) to 146 (K2AC100). This equals a 65%-76% increase in discriminative binding (Example 3, Table 2).

In one embodiment, the first antibody as used in this invention is characterized in a concentration dependent phagocytosis (ADCP of CEACAM5 expressing tumor cell lines by human macrophages). ADCP is measured according to the invention as phagocytosis index (EC50 and/or maximum) by imaging, usually with an E:T ratio of 1:3 (human macrophages:target cells (tumor cells); see e.g. Tables 6 to 9 for EC50 values and for max. index of phagocytosis Emax). Details of the assay are described in Example 7; imaging assay based on CellInsight CX5. If not otherwise stated, phagocytosis index values are measured by such imaging method.

In one embodiment, the first antibody as used in this invention is characterized in an at least 8% increase in the maximum of phagocytosis index (Emax) of LoVo tumor cells in comparison to the phagocytosis index of K2AC22. In one embodiment, the increase is between 8% and 20% for LoVo tumor cells. In one embodiment, the bispecific antibody according to the invention is characterized in an at least 8% increase in the maximum of phagocytosis index of Ls174T tumor cells in comparison to the phagocytosis index of K2AC22. In one embodiment, the increase is between 8% and 25% for Ls174T tumor cells. (Example 7, Table 5). LoVo and LS174T are tumor cells with rather low expression of CEACAM5 (see Table 3 under Example 5).

In one embodiment the first antibody as used in this invention inhibits the interaction between human CD47 and human SIRPα. In one embodiment, the first antibody as used in this invention inhibits the interaction between CD47 and SIRPα on MKN-45 cells with an IC50 which is a factor of 10 or more lower than the IC50 measured for K2AC22 under the same experimental conditions. In one embodiment, said factor is between 10 and 30. In one embodiment, the first antibody as used in this invention inhibits the interaction between CD47 and SIRPα on MKN-45 cells with an IC50 of 0.1 nM or lower. In one embodiment, the first antibody as used in this invention inhibits the interaction between CD47 and SIRPα on MKN-45 cells with an IC50 of 0.1 nM to 0.04 nM (see Example 10 and Table 12).

In one embodiment, the first antibody as used in this invention is characterized in possessing two or more of the following properties: having a ratio of the KD values for the binding to recombinant CEACAM3 and recombinant CEACAM5 of a factor of 100 or more, having a relative uncoupling of binding to CEACAM5 and CEACAM3, having a concentration dependent ADCP, having at least an 8% increase in the maximum of phagocytosis index (Emax) of LoVo tumor cells in comparison to the phagocytosis index of K2AC22, and having the ability to inhibit the interaction between human CD47 and human SIRPα at more than 10 times lower IC50 compared to K2AC22.

Bispecific antibody K2AC22 is a bispecific antibody binding to human CEACAM5 and human CD47 and described in table 1 of WO2019234576. K2AC22 comprises a common heavy chain of SEQ ID NO:5 of WO2019234576, in the CEACAM5 binding part the light chain of SEQ ID NO:65 of WO2019234576, and in the CD47 binding part the light chain of SEQ ID NO:11; CDRs of K2AC22 are shown in SEQ ID NO:1-3, 7-9, and 34-36 of WO2019234576

In one embodiment, the first antibody as used in this invention is characterized in binding to recombinant human CD47 with a binding affinity (KD) of 100 nM to 600 nM, and in one embodiment with a binding affinity of 100 nM to 500 nM (measured by biolayer interferometry).

In one embodiment, the first antibody as used in this invention is characterized in binding to recombinant human CEACAM5 with a KD between 2 nM and 10 nM (Example 3, Table 2). In one embodiment, the first antibody as used in this invention has a 10-fold to 50-fold higher binding affinity (lower KD), and in one embodiment 20-fold to 50-fold, compared to the state-of-the-art bispecific antibody K2AC22 (Example 3, Table 2).

In one embodiment, the first antibody is characterized in specifically binding to CEACAM5 but is not competing with the second antibody as used according to the invention regarding CEACAM5 binding.

The present invention further provides a method of inducing cell lysis of a tumor cell comprising contacting the tumor cell with a bispecific antibody combination according to the invention. The tumor cell is a human tumor cell, in one embodiment in a patient. In one embodiment of a method to induce cell lysis of a tumor cell, the tumor cell is a colorectal cancer cell, NSCLC (non-small cell lung cancer) cell, gastric cancer cell, pancreatic cancer cell, breast cancer cell, or another tumor cell expressing CEACAM5.

The present invention further provides a method of treating a subject having a cancer that expresses CEACAM5, the method comprising administering to the subject a therapeutically effective amount of the bispecific antibody combination according to the invention.

The present invention further provides a method of increasing survival time in a subject having a cancer that expresses CEACAM5, said method comprising administering to said subject a therapeutically effective amount of the bispecific antibody combination according to the invention.

A further embodiment of the invention is such a method according to the invention, characterized in that the cancer is colorectal cancer, non-small cell lung cancer (NSCLC), gastric cancer, pancreatic cancer, or breast.

The present invention further provides a method of treating a subject having a cancer that expresses CEACAM5, the method comprising administering to the subject a therapeutically effective amount of the bispecific antibody combination according to the invention. A further embodiment of the invention is such a method according to the invention, characterized in that the bispecific antibody combination according to the invention is administered in combination with chemotherapy or radiation therapy to a human subject.

The present invention further provides a bispecific antibody combination according to the invention, for use in the manufacture of a medicament for treating a subject having a cancer that expresses CEACAM5. A further embodiment of the invention is a bispecific antibody combination according to the invention, for use in such manufacture of a medicament according to the invention, characterized in that the cancer is selected from the group consisting of colorectal cancer, non-small cell lung cancer (NSCLC), gastric cancer, pancreatic cancer, and breast cancer.

The present invention further provides a bispecific antibody combination according to the invention, for use in simultaneous, separate, or sequential administration in the treatment of a subject having a cancer that expresses CEACAM5. In one embodiment the first bispecific antibody is administered firstly followed by the combination of the first and second bispecific antibody. In one embodiment, the second bispecific antibody is administered first, followed by the combination of the first and second bispecific antibody

A further embodiment of the invention is a bispecific antibody combination according to the invention, for use according to the invention, characterized in that the first bispecific antibody according to the invention and the second bispecific antibody are administered to said subject alternately in 2 to 15 day intervals. In one embodiment the alternate therapy is started with the first antibody. In one embodiment the alternate therapy is started with the second bispecific antibody of the invention.

A further embodiment of the invention is a bispecific antibody combination according to the invention, for use according to the invention, characterized in that the first bispecific antibody and the second bispecific antibody are administered to said subject simultaneously in 2 to 15 day intervals.

A further embodiment of the invention is a bispecific antibody combination according to the invention, for use according to the invention, characterized in that said cancer is colorectal cancer, non-small cell lung cancer (NSCLC), gastric cancer, pancreatic cancer, and breast cancer.

A further embodiment of the invention is a method for the treatment of a human patient diagnosed with a tumor (cancer), especially a solid tumor, especially a solid cancer that expresses CEACAM5 especially colorectal cancer, non-small cell lung cancer (NSCLC), gastric cancer, pancreatic cancer, and breast cancer, comprising administering an effective amount of the bispecific antibody combination according to the invention to the human patient, the method comprising subsequently: administering the first bispecific antibody to the patient a dose of 0.1 to 10 mg/kg, in a further embodiment of 0.5 to 10 mg/kg, in a further embodiment of 10 to 30 mg/kg and of 0.1 to 1 mg/kg, in a further embodiment of 1 to 10 mg/kg of the second bispecific antibody, e.g. weekly over 4 to 12 weeks or q2w, over 4 to 12 weeks and administering after these 4 to 12 weeks and after waiting for additional 2 or 3 or 4 elimination half-lives of the second bispecific antibody to the patient a dose of 0.1 to 20 mg/kg of the first bispecific antibody,

administering to the patient said first antibody q1, q2w, q3w or optionally q4w for e.g., 12 more weeks, waiting 2 or 3 or 4 elimination half-lives of the first bispecific antibody and then optionally repeating said cycle of administration of the second bispecific antibody followed by administration of the first bispecific antibody and optionally repeat again that cycle etc.

As the CEA×CD3 bispecific antibody (AB73) and the CEA×CD47 bispecific antibody (K2AC100) are not competitive for binding to CEACAM5, the two bispecific antibodies can also be administered in a manner (“simultaneous manner”) that the patient experiences therapeutically effective plasma and tissue concentrations of both bispecific antibodies in parallel, e.g. by administration to the patient at about the same time a dose of 0.1 to 10 mg/kg, in a further embodiment of 0.5 to 10 mg/kg, in a further embodiment of 10 to 20 mg/kg of the first bispecific antibody and a dose of 0.1 to 10 mg/kg in a further embodiment of 1 to 10 mg/kg of the second bispecific antibody according to the invention, followed by one or more of these combined administrations at a frequency of q1w or q2w or q3w or optionally q4w. The term “qlw” means administration once a week; q2w means administration every two weeks etc.

For safety reasons it may be needed in one embodiment to start the therapy with the said second bispecific antibody w/o adding the first bispecific antibody and to follow afterwards with simultaneous administration of the two bispecific antibodies.

The present invention further provides a pharmaceutical composition comprising the bispecific antibody combination according to the invention and a pharmaceutically acceptable excipient or carrier.

The present invention further provides a pharmaceutical composition comprising the bispecific antibody combination according to the invention for use as a medicament. In one such embodiment the present invention provides a pharmaceutical composition comprising the bispecific antibody combination according to the invention for use as a medicament in the treatment of solid tumor disorders. In one embodiment, the pharmaceutical composition comprises the bispecific antibody combination according to the invention for use as a medicament in the treatment of colorectal cancer, NSCLC (non-small cell lung cancer), gastric cancer, pancreatic cancer, or breast cancer.

The present invention further provides a composition comprising the bispecific antibody combination according to the invention, for use in simultaneous, separate, or sequential combination in the treatment of a subject having a cancer that expresses CEACAM5.

The present invention further provides the use of the bispecific antibody combination according to the invention for the manufacture of a pharmaceutical composition.

The present invention further provides use of the bispecific antibody combination according to the invention and a pharmaceutically acceptable excipient or carrier for the manufacture of a pharmaceutical composition.

The present invention further provides use of the bispecific antibody combination according to the invention for the manufacture of a medicament in the treatment of solid tumor disorders. A further embodiment of the invention is such use of the bispecific antibody combination according to the invention in the treatment of colorectal cancer, NSCLC (non-small cell lung cancer), gastric cancer, pancreatic cancer, or breast cancer and other CEACAM5 expressing cancers.

Another aspect of the invention provides a method of inducing cell lysis of a tumor cell comprising targeting the tumor cell with the bispecific antibody combination of any of above described embodiments. In some embodiments, the tumor cell is a colorectal cancer cell, NSCLC (non-small cell lung cancer), gastric cancer cell, pancreatic cancer cell or breast cancer cell. In one embodiment, the cell lysis is induced by antibody dependent cellular phagocytosis and/or antibody dependent cell mediated cytotoxicity of the bispecific antibody combination according to the invention. In one embodiment, the tumor cell lysis is induced by macrophage induced phagocytosis and T-cell activation.

Another aspect of the invention provides a method of treating a subject having a cancer that overexpresses CEACAM5, the method comprising administering to the subject a therapeutically effective amount of the bispecific antibody combination of any of above-described embodiments.

Another aspect of the invention provides a method of treating a subject having a cancer that overexpresses CEACAM5, the method comprising administering to the subject a therapeutically effective amount of the bispecific antibody combination of any of above-described embodiments.

As the CEA×CD3 bispecific antibodies and the CEA×CD47 bispecific antibodies are not or only minimally competing, they can not only be given sequentially, but likewise in parallel (simultaneously), which may well be an advantage because tumor cell killing via engagement of T-cells by the CEA×CD3 bispecific antibody and at the same time via engagement of macrophages by the CEA×CD47 bispecific antibody according to the invention may be additive or even synergistic, which means efficacy is increased if both bispecific antibodies are given in parallel.

Another aspect of the invention provides a method of increasing progression free survival and/or overall survival time in a subject having a cancer that overexpresses CEACAM5, said method comprising administering to said subject a therapeutically effective amount of the bispecific antibody combination of any of above described embodiments. In one embodiment, the cancer is colorectal cancer, non-small cell lung cancer (NSCLC), gastric cancer, pancreatic cancer or breast cancer or any other cancer expressing CEACAM5.

In certain embodiments of these methods, the bispecific antibody combination according to the invention is administered in combination with chemotherapy or radiation therapy. In one embodiment, the subject is a patient suffering from colorectal cancer or lung cancer or gastric cancer or pancreatic cancer or breast cancer or another cancer expressing CEACAM5.

Another aspect of the invention provides a method of treating a subject having a cancer that overexpresses CEACAM5, the method comprising administering to the subject a therapeutically effective amount of the bispecific antibody of any of above-described embodiments in combination with a bispecific antibody against human CEA and human CD3epsilon.

Another aspect of the invention provides a method of increasing progression free survival time and/or overall survival time in a subject having a cancer that overexpresses CEACAM5, said method comprising administering to said subject a therapeutically effective amount of the bispecific antibody combination of any of above-described embodiments. In one embodiment, the cancer is colorectal cancer, non-small cell lung cancer (NSCLC), gastric cancer, pancreatic cancer, or breast cancer.

In certain embodiments of these methods, the bispecific antibody combination according to the invention is administered in combination with chemotherapy or radiation therapy. In one embodiment, the subject is a cancer patient with colorectal cancer or lung cancer or gastric cancer or pancreatic cancer or breast cancer or another CEACAM5 expressing cancer.

Another embodiment of the invention provides the use of a bispecific antibody combination according to the invention for any of the above-described methods of treatment. In one embodiment, the cancer is selected from the group consisting of colorectal cancer, non-small cell lung cancer (NSCLC), gastric cancer, pancreatic cancer, and breast cancer.

### BRIEF DESCRIPTION OF THE FIGURES

FIG. 1. Killing achieved in a mixed assay (MKN-45 cells).

FIG. 1 shows the concentration response curves for AB73 (CEA×CD3 bispecific antibody) in monotherapy and in combination with 0.1, 1 and 10 μg/ml of K2AC100 (CEA×CD47 bispecific antibody) for MKN-45 cells. Even at the highest concentration of AB73 monotherapy, only slightly above 30% of killing was achieved. Addition of 0.1 μg/ml K2AC100 increased maximal killing to approximately 40%, addition of 1 or 10 μg/kg increased killing to approximately 80%. Killing by the combination started at much lower concentrations of AB73 compared to killing at AB73 monotherapy; hIgG1=control (Description of mixed assay see Example 11).

FIG. 2. Data from the same study in a mixed assay from which results are shown in FIG. 1.

FIG. 2A shows % killing achieved with 1 g/ml K2AC100 combined with 0.6, 0.12 or 0.024 g/ml AB73 in monotherapy and in combination; FIG. 2B shows the results if 10 g/ml K2AC100 were used instead of 1 g/ml; higher % of killing always achieved with the combination compared to the two monotherapies.

FIG. 3. Killing achieved in a mixed assay (LS-174T cells).

FIG. 3 shows the concentration response curves for AB73 in monotherapy and in combination with 0.1, 1 or 10 g/ml of K2AC100. Monotherapy with AB73 achieved at the highest concentrations tested already approximately 70% killing. Addition of K2AC100 (0.1, 1, 10 g/ml) shifted the concentration response curve of AB73 to the left, a maximum of approximately 80% killing was achieved; hIgG1=control (Description of mixed assay see Example 11).

FIG. 4. Data from the same study in a mixed assay from which results are shown in FIG. 3.

FIG. 4A shows % killing achieved with 1 μg/ml K2AC100 in monotherapy or combined with 0.08, 0.016 or 0.0032 μg/ml AB73; FIG. 4B shows the results if 10 μg/ml K2AC100 were used instead of 1 μg/ml and same concentrations of AB73 as under FIG. 4A; with the combination higher % of killing was always achieved compared to the two monotherapies.

FIG. 5. Killing achieved in a mixed assay (LS-174T cells).

FIG. 5 shows the concentration response curves for K2AC100 in monotherapy and in combination with 5 μg/ml of AB73. Monotherapy with K2AC100 achieved at the highest concentrations tested already above 90% killing. Addition of AB73 (5 μg/ml) shifted the concentration response curve of K2AC100 to the left; hIgG1=control (Description of mixed assay see example 11).

FIG. 6. Data from the same study from which results are shown in FIG. 5.

FIG. 6 shows % killing achieved with 5 μg/ml AB73 in monotherapy and for the combination of 5 μg/ml AB73 with 0.4 or 0.08 μg/ml K2AC100; higher % of killing always achieved with the combination compared to the two monotherapies.

### DETAILED DESCRIPTION OF THE INVENTION

Due to potential side effects like Cytokine Release Syndrome CRS (reported for TAA×CD3) etc. compared to higher dose therapy with only one of the bispecific antibodies, it is beneficial if the same maximal killing of tumor cells can be achieved by lower drug concentrations. Unexpectedly, the combination according to the invention shows such advantages:

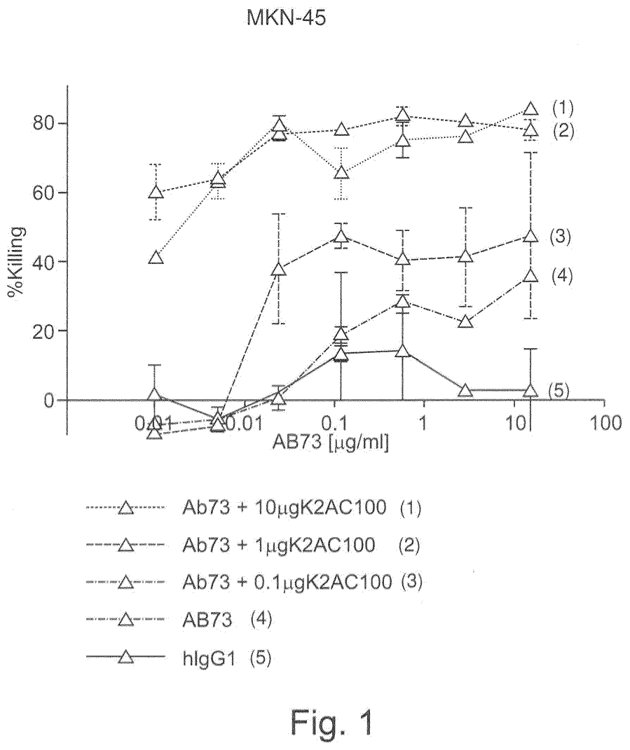

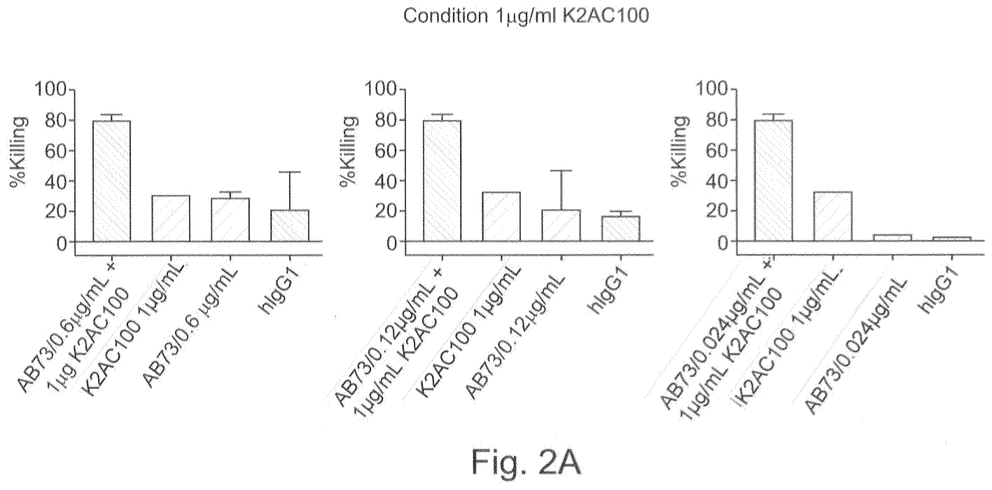

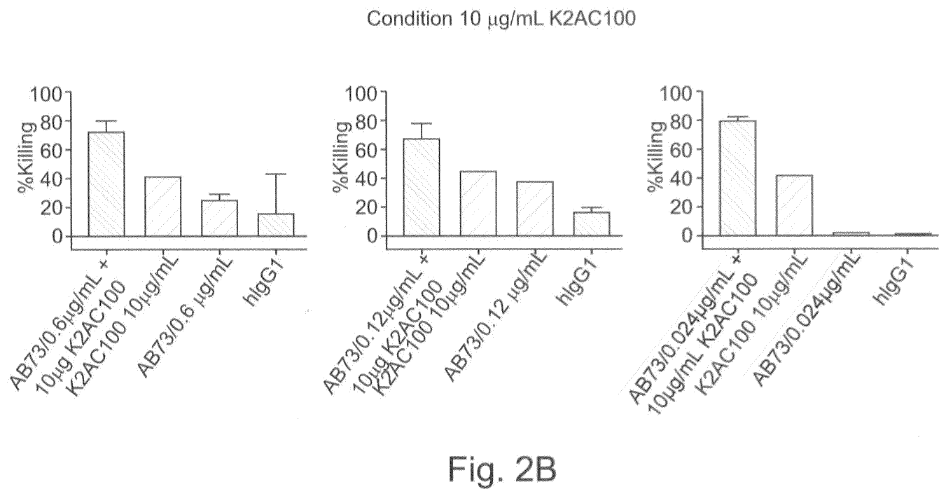

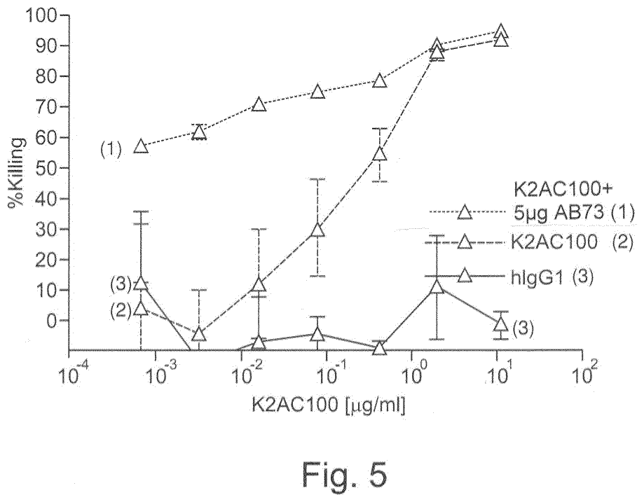

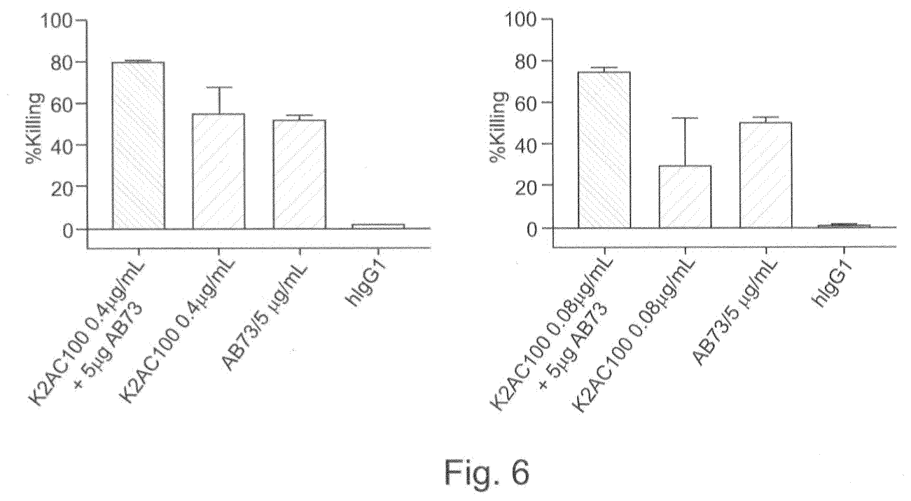

The combination according to the invention increases in a mixed assay using human macrophages and PBMC/T-cells from the same donor the maximal killing of tumor cells substantially.
The combination of the invention increases in a mixed assay using human macrophages and PBMC/T-cells from the same donor the maximal killing of tumor cells like MKN-45 cells from approximately 30% killing at monotherapy to approximately 80% killing at combination (FIG. 1, Example 11).
The combination of the invention shows in a mixed assay using human macrophages and PBMC/T-cells from the same donor at all the concentrations tested higher % of killing of tumor cells like LS-174T and/or MKN-45 in the combination compared to the monotherapies if the same concentrations are used (FIGS. 2, 4, 6, Example 11).
The combination of the invention of a CEA×CD47 bispecific antibody with a CEA×CD3 bispecific antibody shows in a mixed assay using human macrophages and PBMC/T-cells from the same donor high % of killing (>80%) in monotherapy of one of the bispecific antibodies as well as in combination, but if lower concentrations are tested the % of killing is superior in combination compared to monotherapies (FIG. 5 and FIG. 6, Example 11).

Terms are used herein as generally used in the art, unless otherwise defined as follows.

The term “as used” or “as used according to the invention” as used herein means “as used in the combination or in the method according to the invention”.

The term “AB73L3-1/N” as used herein is characterized as a bispecific CEA×CD3 antibody, comprising the common heavy chain CDRs of SEQ ID NOs: 2, 3, and 4, and in the first binding part to CEA the light chain CDRs of SEQ ID NOs: 18, 19, and 20 and in the second binding part to CD3 the light chain CDRs of SEQ ID NOs: 6, 7, and 8. The AB73 part denotes for a first binding part (anti-CEACAM5 binding part) and L3-1 denotes for a second binding part (anti-CD3 binding part) of said antibody. In one embodiment AB73L3-1/N comprise a common heavy chain of SEQ ID NO: 15 (IgG1 with L234A+L235A+P329A mutations), in the first binding part the light chain of SEQ ID NO: 22 and in the second binding part the light chain of SEQ ID NO: 9.

The term “AB73”, as used herein, denotes said AB73L3-1/N antibody and is a kappa lambda CEA×CD3 bispecific antibody.

The term “K2AC100” as used herein, is characterized as a bispecific CEA×CD47 antibody comprising the common heavy chain CDRs of SEQ ID NOs: 23, 24 and 25 and in the first binding part to CEA the light chain CDRs of SEQ ID NOs: 35, 36 and 37 and in the second binding part to CD47 the light chain CDRs of SEQ ID NOs:29, 30 and 31. AC100 denotes for a first binding part (anti-CEACAM5 binding part) and K2 denotes for a second binding part (anti-CD47 binding part) of said antibody.

In one embodiment K2AC100 comprises a common heavy chain of SEQ ID NO:28 (IgG1 WT), in the first binding part to CEA the light chain of SEQ ID NO: 39 and in the second binding part to CD47 the light chain SEQ ID NO: 32.

The term “first antibody” as used herein means a bispecific antibody against CEACAM5 and CD47 (CEA×CD47 bispecific antibody; CEA×CD47 antibody, K2AC100) as defined herein. The term “second antibody” as used herein means a bispecific antibody against CEACAM5 and CD3 (CEA×CD3 bispecific antibody; CEA×CD3 antibody; AB73) as defined herein. The term “both antibodies” or “the antibodies” as used herein means “the first and the second antibody”.

The term “antibody according to the invention” and “antibody as used according to the invention”, as used herein, means “antibody as used in the combination or in the method according to the invention”.

The term “combination treatment, co-administration, used in combination, combination of the first and second antibody” as used herein, means that the first and second antibody are used, formulated, administered simultaneously or subsequently. Details for such use, treatment and combinations are described in detail below.

The CEA×CD47 antibody as used according to the invention has one or more beneficial properties out of the following properties:
ratio of KD values for the binding to CEACAM3 vs. CEACAM5,
increase of maximum of phagocytosis index (Emax) in low CEA expressing tumor cells, and/or
inhibition at a low IC50 of the binding of SIRPα to CD47 on the surface of tumor cells.

Quite unexpectedly, the first bispecific antibody has a relative uncoupling of binding to CEACAM5 and CEACAM3 (discriminative binding) and increased binding to CEACAM5 does not result in increase of binding to CEACAM3 proportionally (Example 3 and Table 2). For example, K2AC100 shows a 25-fold higher binding affinity (lower KD) to CEACAM5 but surprisingly only a 14-fold higher binding affinity (lower KD) to CEACAM3, compared to the binding affinities (KD) of K2AC22. Thus, the ratio of the KD value for the binding to CEACAM3 to the KD value for the binding to CEACAM5 is 83 for K2AC22, but is 146 for K2AC100.

While several family members like CEACAM5 or CEACAM6 are expressed by epithelial cells, other family members, such as CEACAM3 (CGM1 or CD66d; UniProtKB—P40198), are exclusively expressed on human granulocytes, a cell type e.g. involved in the clearance of bacterial infection (Kuespert K et al., Curr Opin Cell Biol. 2006; Pils S et al., Int J Med Microbiol. 2008). Despite the high sequence-homology between CEACAM5 and CEACAM3, CEACAM3 does not support cell-cell adhesion in contrast to other members of the CEACAM family, but rather mediates the opsonin-independent recognition and elimination of a restricted set of Gram-negative bacteria including Neisseria gonorrhoeae, Hemophilus influenzae, and Moraxella catarrhalis (Kuroki et al., J. Biol. Chem. 1991; Pils S et al., Int J Med Microbiol. 2008). CEACAM3 is discussed as phagocytic receptor of the innate immune system (Schmitter et al., J Exp Med. 2004). According to the knowledge of the inventors a bispecific antibody against CEACAM5 and CD47, if considerably binding also to CEACAM3, would have an adverse effect on neutrophil granulocytes and could decrease the numbers of neutrophils, i.e. induce neutropenia by increased phagocytosis. This could increase the risk of developing bacterial infections which can, without immediate medical intervention, become life-threatening, especially in cancer patients with often deficient immune system. High binding affinity is characterized by a low KD. The distribution of a CEA targeting bispecific antibody between CEACAM 5 and CEACAM3 is determined by the ratio of the binding affinities to these two CEACAM family members. A high ratio of the KD for binding to CEACAM3 versus the KD for the binding to CEACAM5 means less binding of the bispecific antibody to CEACAM3 compared to binding to CEACAM5, which would be beneficial.

Quite unexpectedly, the CEA×CD47 bispecific antibody, shows an increase in Emax for phagocytosis compared to K2AC22 in the low CEACAM5 expressing cell lines LS174T and LoVo, whereas there is no increase in Emax for high CEACAM5 expressing cell line SNU-C1. Therefore, the first antibody shows surprisingly a beneficial increase of the maximum value of phagocytosis index (Emax) in low CEA expressing tumor cells (like the LoVo cell line) in comparison to the increase of the phagocytosis index achieved with K2AC22 in the respective cell lines. According to Table 5 the first antibody shows an 8.5 to 17% higher increase of the maximum of the phagocytosis index curve of LoVo cells (4000 CEACAM5 on cell surface) compared to the bispecific antibody of the state of art K2AC22. For LS174T cells (26000 CEACAM5 on cell surface) the increase of the maximum of the phagocytosis index is between 8.7 to 20.6% higher for the antibodies of the invention compared to K2AC22 (Table 5). In higher CEACAM5 expressing cells like SNU-C1 or MKN-45 the increase of Emax is lower or there is even no increase compared to K2AC22.

A higher percentage of patients could be therefore successfully treated with bispecific antibodies according to the invention.

As disclosed in Examples 5 and 11, CEA expression in malignant cells can vary significantly in terms of RNA-expression or enumeration of cell surface CEA molecules. The CEA expressing cancer cell lines used to study phagocytosis activity of the bispecific CEA×CD47 antibodies express on average 108,000 CEA targets on the cell surface (Example 5, Table 3). Organoids derived from fresh tumor tissue of cancer patients (colorectal and lung) have been investigated by the methods described in Example 10. The average expression of CEACAM5 of these primary organoids has been found as 28,000 CEACAM5 targets per cell, i.e. a factor of approximately 4 lower than average expression on the cell lines as shown in Table 3. The first antibody shows therefore improved phagocytosis of malignant cells with lower CEACAM5 expression. This could thus be favorable for use in tumor therapy. Given the heterogenous and/or rather low expression e.g. in lung adenocarcinoma, in colorectal cancer and other CEACAM5 expressing tumors, such patients may be therefore successfully treated with the CEA×CD47 bispecific antibody of this invention.

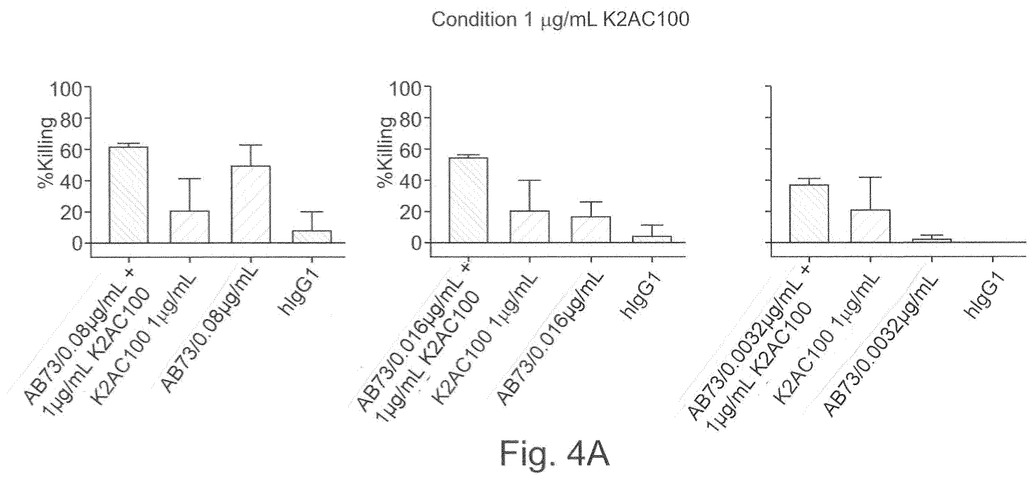

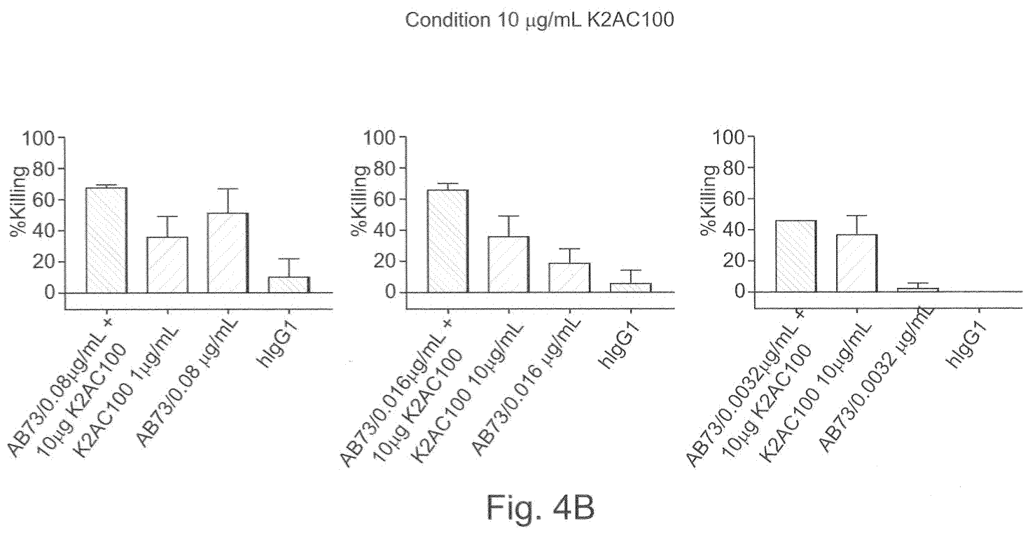

The first antibody shows surprisingly a beneficial inhibition (low IC50) of the binding of SIRPα to CD47 on the surface of tumor cells in comparison to antibody K2AC22, as shown in Example 9 and FIG. 4. The interaction of SIRPα on macrophages with CD47 on tumor cells inhibits the phagocytosis of the tumor cells, that means effective inhibition of this interaction increases phagocytosis.

As used herein the term “Emax” describes the maximal activity of a compound. For example, in a cell killing assay, an Emax describes the % elimination/killing of cancer cells (e.g. labelled with calcein AM, see Example 7) by macrophages within a given timeframe at concentrations which are already in saturation. This is of presumed high clinical importance as the total number of tumor infiltrating macrophages is limited: if for example double the number of tumor cells are eliminated per time interval, this equals to half the number of macrophages needed to be present to eliminate the same number of tumor cells per time.

As used herein the term “EC50” describes the compound concentration at which half of maximal activity (Emax/2) is reached. A low EC50 is useful in order to need to infuse a lower amount of compound and therefore to achieve e.g. lower production cost compared to a higher EC50 and/or potentially but not necessarily also lower rate of side effects. Emax and EC50 therefore describe different aspects of compound activity. For two compounds of comparable Emax, the EC50 becomes important as the same therapeutic effect could be achieved at a lower concentration and thus less amount of drug to be given and potentially lower rate of side effects to be achieved.

As used herein, the terms “antigen binding part” and “binding part” refer in their broadest sense to a part of an antibody that specifically binds an antigenic determinant such as CEA, CD47 and CD3. The binding part comprises therefore the six CDRs of the heavy and light chain, which are part of the light and heavy variable chains, and part of the light and heavy chains

More specifically, as used herein, a binding part that connects membrane-bound human carcinoembryonic antigen (CEA, same as CEACAM5), to CD47 or to CD3 specifically binds to CEA, CD47 or CD3, more particularly to cell surface or membrane-bound CEA, CD47 or CD3. Therefore, each binding part binds either to CEA, CD47 or CD3. By “specifically binding, specific for, binding to” is meant that the binding is selective for the antigen and can be discriminated from unwanted or nonspecific interactions. In some embodiments, the extent of binding of an anti-target antibody to an unrelated, non-target protein is about 10-fold, preferably >100-fold less than the binding of the antibody to said target as measured, e.g., by biolayer interferometry e.g. Octet®, surface plasmon resonance (SPR) e.g. Biacore®, enzyme-linked immunosorbent (ELISA) or flow cytometry (FACS). Targets are the proteins discussed herein—e.g. CEA, CD47, and CD3ε.

The phrases specifically binding to CEA and CD47, binding to CEA and CD47, and specific for CEA and CD47 refer in one embodiment to an antibody, e.g., bispecific antibody, that is capable of binding to the targets CEA and CD47 with sufficient affinity such that the antibody is useful as a therapeutic agent in targeting tumor cells expressing CEACAM5 and CD47. Reference to binding to MKN-45, SNU-C1, LS174T, SK-CO-1, HPAF-II and/or LoVo cells with a particular EC50 value refers to an EC50 value measured by flow cytometry (see Example 6).

As used herein, the term “antibody” refers to an antibody comprising two heavy chains and two light chains. In one embodiment, the antibody is a full-length antibody. As used herein, the term “antibody heavy chain” refers to an antibody heavy chain, consisting of a variable region and a constant region as defined for a full-length antibody. As used herein, the term “antibody light chain” refers to an antibody light chain, consisting of a variable region and a constant region as defined for a full-length antibody.

The term “full-length antibody” denotes an antibody consisting of two “full-length antibody heavy chains” and two “full-length antibody light chains”. A “full-length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3. A “full-length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full-length antibody domains are linked together via inter-polypeptide disulphide bonds between the CL domain and the CH1 domain and between the hinge regions of the full-length antibody heavy chains. Examples of typical full-length antibodies are natural antibodies like IgG (e.g. IgG 1 and IgG2), IgM, IgA, IgD, and IgE. The full-length antibody according to the invention is in one embodiment of human IgG1 type, in one further embodiment comprising one or more amino acid substitutions in the Fc part as defined below and/or being glycoengineered at polysaccharide chain attached to Asn297. The full-length first antibody according to the invention comprise two binding parts each formed by a pair of VH and VL, one binding to CEA and the other binding to CD47. The full-length second antibody according to the invention comprise two binding parts each formed by a pair of VH and VL, one binding to CEA and the other binding to CD3.

As used herein and mentioned above, “Complementarity determining region(s)” (CDR(s)) describe the non-contiguous antigen combining sites (also known as antigen binding regions) found within the variable region of both heavy and light chain polypeptides. CDRs are also referred to as “hypervariable regions” (HVRs), and that term is used interchangeably herein with the term “CDR” in reference to the portions of the variable region that form the antigen binding regions. This particular region has been described by Kabat et al., Sequences of Proteins of Immunological Interest, 5th Ed. Public Health Service, National Institutes of Health, Bethesda, MD (1991); which is incorporated herein by reference. The appropriate amino acid residues which encompass the CDRs as defined by Kabat are set forth below in the sequence list table. The exact residue numbers which encompass a particular CDR will vary depending on the sequence and size of the CDR. Those skilled in the art can routinely determine which residues comprise a particular CDR given the variable region amino acid sequence of the antibody. As used herein the term “comprising a CDRL1 of SEQ ID NO: x” refers to that the CDRL1 region of the referred variable light chain is of SEQ ID NO: x (comprising as CDRL1 a CDRL1 of SEQ ID NO: x). This is true also for the other CDRs. Unless otherwise indicated, HVR residues are numbered herein according to Kabat et al., supra and named as “CDRs” and references to the numbering of other specific amino acid residue positions in the bispecific antibodies according to the invention are also according to the Kabat numbering system.

As used herein, the terms “Fc region” and “Fc domain” refer to a C-terminal region of an IgG heavy chain; in case of an IgG1 antibody, the C-terminal region comprises —CH2-CH3 (see above). Although the boundaries of the Fc region of an IgG heavy chain might vary slightly, the human IgG heavy chain Fc region is usually defined to stretch from the amino acid residue at position Cys226 to the carboxyl-terminus. Constant regions are well known in the state of the art and e.g. described by Kabat, E. A., (see e.g. Johnson, G., and Wu, T. T., Nucleic Acids Res. 28 (2000) 214-218; Kabat, E. A., et al, Proc. Natl. Acad. Sci. USA 72 (1975) 2785-2788).

An IgG molecule carries two N-linked oligosaccharides in its Fc region, one on each heavy chain. As any glycoprotein, an antibody is produced as a population of glycoforms which share the same polypeptide backbone but have different oligosaccharides attached to the glycosylation sites. Antibodies with a reduced fucose content in glycan moieties exhibit higher antibody-dependent cellular cytotoxicity (ADCC) activity compared to a normally fucosylated antibody (Niwa R et al., Cancer Res, 64, 2127-33, 2004). A cell line with knockout of both alleles for the gene responsible for fucose addition (α1,6-fucosyltransferase; FUT8) is described in U.S. Pat. Nos. 6,946,292, 7,425,446, 8,067,232 (each of which is incorporated by reference in its entirety). Using such a cell line the bispecific antibodies according to the invention can be produced with glycan moieties having a reduced fucose content and increased ADCC and antibody-dependent cellular phagocytosis (ADCP). Another technology which can be used to produce antibodies with reduced fucose content is described in U.S. Pat. No. 8,642,292 (incorporated herein by reference). This technology is designed to configure the stable integration of a heterologous bacterial enzyme into an antibody producer cell line like a CHO cell line or others. By this measure, the de novo synthesis of fucose from D-mannose is blocked. If in addition production cells are cultivated in fucose free medium, as a result antibodies with a stable level of afucosylation are produced. An exemplary method to produce and purify the afucosylated bispecific antibodies of this invention is described in Example 9 (1. and 2).

Mutations within the Fc domain can also alter binding properties of the Fc domain to the different Fc receptors (WO2004063351, WO2004099249; WO2005018669, WO2005063815, WO2005110474, WO2005056759, WO2005092925, WO2005018572, WO2006019447, WO2006116260, WO2006023420, WO2006047350, WO2006085967, WO2006105338, WO2007021841, WO2007008943, WO2007024249, WO2007041635, WO2007048077, WO2007044616, WO2007106707, WO2008022152, WO2008140603, WO2008036688, WO2008091798, WO2008091954, WO2008092117, WO2008098115, WO2008121160, WO2008150494, WO2010033736, WO2014113510 (each of which is incorporated by reference in its entirety)).

The term “epitope” includes any polypeptide determinant capable of specific binding to an antibody. In certain embodiments, “epitope” includes chemically active surface groupings of molecules such as amino acids, sugar side chains, phosphoryl, or sulfonyl, and, in certain embodiments, may have specific three-dimensional structural characteristics, and or specific charge characteristics. An epitope is a region of a target that is bound by an antibody. In one embodiment, the first bispecific antibody bind to the N-terminal domain of CEACAM5 (Ig-like V-type domain of amino acids 35-144, UniProtKB—P06731). Binding location of the bispecific antibodies to CEACAM5 is achieved via epitope binning. In epitope binning, antibodies are tested in a pairwise combinatorial manner, and antibodies that compete for the same binding region are grouped together into bins. Competition testing is performed herein with anti-CEA antibodies according to the state of the art and as described herein. In one embodiment, the first bispecific antibody of the invention competes for binding to CEACAM5 with reference antibody SM3E. Competition is measured by an assay wherein biotinylated human CEACAM5 in a concentration of 0.5 μg/ml is immobilized and incubated with serial dilution (from 67 nM to 0.09 nM) of the reference. Bispecific antibodies of the present invention are added at 0.1 μg/ml for 1 hour at room temperature. The plate is washed and the bound CEA×CD47 or CEA×CD3 bispecific antibodies are detected.

As used herein, the term “a common heavy chain” (cHC) refers to a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH-HR-CH2-CH3. Common heavy chains suitable for the bispecific antibodies according to the invention are heavy chains as described in WO2012023053, WO2013088259, WO2014087248, and WO2016156537 (each of which is incorporated by reference in its entirety). In one embodiment, common heavy chain of the first antibody comprises as heavy chain CDRs a CDRH1 of SEQ ID NO:23, a CDRH2 of SEQ ID NO:24 and a CDRH3 of SEQ ID NO:25. In one embodiment, the cHC of the bispecific antibodies according to the invention comprises as heavy chain variable region VH a VH region of SEQ ID NO:26. In one embodiment, the Fab part of the common heavy chain cHC of the bispecific antibodies according to the invention is of SEQ ID NO:27 (VH-CH1). In one embodiment, the common heavy chain cHC of the bispecific antibodies according to the invention is of SEQ ID NO:28 (VH-CH1-CH2-CH3). SEQ ID NO:28, is a heavy chain comprising in addition an IgG1 Fc part. In one embodiment, the antibodies according to the invention are d, bispecific antibodies comprising a cHC (d, Body).

The d, Body format allows the affinity purification of bispecific antibodies with characteristics that are undistinguishable from a standard monoclonal antibody (see e.g. WO2013088259, WO2012023053), promising no or low immunogenicity potential in patients.

Bispecific antibodies a used according to the invention, comprise a common heavy chain, can be made for example according to WO2012023053 (incorporated by reference in its entirety). This type of molecule is composed of two copies of a unique heavy chain polypeptide, a first light chain variable region fused to a constant Kappa domain and second light chain variable region fused to a constant Lambda domain. One binding site displays specificity to CEA and the other site displays specificity to CD47, wherein to each the heavy and the respective light chain contribute. The light chain variable regions can be of the Lambda or Kappa family and are preferably fused to a Lambda and Kappa constant domains, respectively. This is preferred in order to avoid the generation of non-natural polypeptide junctions. However, it is also possible to obtain bispecific antibodies of the invention by fusing a Kappa light chain variable domain to a constant Lambda domain for a first specificity or fusing a Lambda light chain variable domain to a constant Kappa domain for the second specificity. The other light chain is then always fully kappa (VL and CL) or fully lambda (so called hybrid formats of kappa lambda bispecific antibodies). The bispecific antibodies described in WO 2012023053 are “d, Bodies”. This κλ-Body format allows the affinity purification of a bispecific antibody that is undistinguishable from a standard IgG molecule with characteristics that are undistinguishable from a standard monoclonal antibody and, therefore, favourable as compared to previous formats including e.g. amino acid bridges or other un-physiological elements.

As used herein, the terms “CEA” and “CEACAM5” refer to human carcinoembryonic antigen (CEA, CEACAM-5 or CD66e; UniProtKB—P06731) which is a cell surface glycoprotein and a tumor-associated antigen (Gold and Freedman, J Exp. Med., 121:439-462, 1965; Berinstein N L, J Clin Oncol., 20:2197-2207, 2002). As used herein, the term “CEACAM3” refers to human CEACAM3 (UniProtKB—P40198 (CEAM3 HUMAN) which is also a member of the carcinoembryonic antigen-related cell adhesion molecule (CEACAM) family. Further information and information on other members of the CEA family can be found under http://www.uniprot.org.

As used herein, the terms “specifically binding to CD47,” “binding to CD47,” and “CD47 binding part” refer in the context of the bispecific antibodies according to the invention to specificity for human CD47. Human CD47 is a multi-pass membrane protein and comprises three extracellular domains (amino acids 19-141, 198-207, and 257-268; see UniProtKB—Q08722). As used herein the “binding affinity to CD47” is measured quantitatively (KD) by biolayer interferometry (Octet Technology) and/or surface plasmon resonance (Biacore Technology). In one embodiment, binding of the first bispecific antibody according to the invention to CD47 occurs via one or more of said extracellular domains.

As used herein, the term “characterized by a heavy chain of SEQ ID NO:27” refers, as shown in Table 1, to the VH-CH1 part of the heavy chain which is the Fab part of the antibody according to the invention. Such heavy chain can comprise in addition, and according to common knowledge, further parts as hinge region, CH2, CH3, and can be in any antibody format, like in the F(ab′)₂ format. The preferred format is the common heavy chain format as described above

As used herein, the terms “specifically binding to CEA,” “binding to CEA,” and “CEA binding part” refer to binding of a bispecific antibody according of the invention to recombinant human CEACAM5, wherein said antibody binds to recombinant human CEACAM3 with a KD value of 100-fold or higher compared to the KD value of the binding to recombinant human CEACAM5.

The term “KD”, as used herein, refers to the equilibrium dissociation constant between the bispecific antibody according to the invention and its antigen CEACAM5 or CEACAM3 and is specified in nM and can be e.g. measured by surface plasmon resonance and/or biolayer interferometry (Example 3).

Binding to CEA (CEACAM5) on cells is measured by using different tumor cell lines like LoVo, LS174T, MKN-45, SNU-C1, SK-CO-1, HPAF-II. The concentration of the first antibody according to the invention is varied in an appropriate range regarding a resulting EC50 value and Emax value for binding to cells as defined above. EC50 and Emax for binding of K2AC22 and K2AC100 to various tumor cell lines are listed in Table 4.

As used herein, the term “membrane-bound human CEA” refers to human carcinoembryonic antigen (CEA) that is bound to a membrane-portion of a cell or to the surface of a cell, in particular, the surface of a tumor cell.

As used herein “CD3ε” and “CD3” refer to human CD3ε (UniProtKB—P07766 (CD3E_HUMAN). The terms “antibody against CD3ϵ (CD3)” and “anti CD3ε (CD3) antibody” relate to an antibody that specifically binds to CD3ε. In one embodiment, the antibody against CD3ε specifically binds to the same epitope as anti-CD3 antibody SP34 (BD Biosciences Catalog No. 565983).

As used herein the term “ADCP” refers to antibody-dependent cellular phagocytosis. As used herein phagocytosis, EC50 value of phagocytosis, maximum of phagocytosis, and phagocytosis index according to the invention refer to phagocytosis measured with tumor cell lines like e.g. LoVo, LS174T, SNU-C1 and/or MKN-45 by “imaging.” An appropriate imaging method, with incubation at an effector (macrophages):target (tumor) cell ratio of e.g. 1:1 or 1:3 and with the “phagocytosis index” as readout (imaging determined ADCP”) is described in Example 7. As used herein “phagocytosis of said bispecific antibody” means phagocytosis caused/induced by said antibody.

The terms “human IgG” and “hIgG” refer to a human antibody isotype. As used in experimental setups, these terms refer to a commercially available clinical-grade homogeneous preparation of human immunoglobulin IgG (available e.g. from Bio-Rad) that does not bind specifically to CD47 and CEACAM5.

As used herein “antibody K2AC22” refers to the antibody as disclosed in WO2019234576. The antibody comprises a common heavy chain (SEQ ID NO:5 of WO2019234576), a light chain binding to CEACAM5 (SEQ ID NO:65 of WO2019234576), and a light chain binding to CD47 (SEQ ID NO:11 of WO2019234576).

### Therapeutic Applications and Methods of Using Anti-CEA Antigen Binding Molecules

The combination therapy according to the invention is optimized for treatment of solid tumors mainly by macrophages mediated phagocytosis of the tumor cells, but also by ADCC, in one embodiment in combination with a PD-1 axis antagonist. The antibodies as used according to the invention can be administered as described below.

In a particular embodiment, the disease resp. solid tumor is a cancer that expresses or even overexpresses CEACAM5, including but not limited to the group of colorectal tumors, non-small cell lung tumors, gastric tumors, pancreatic tumors, and breast tumors. In a particular embodiment, the tumor is a colorectal tumor. In a particular embodiment the tumor is a gastric tumor or a gastroesophageal junction tumor. In a particular embodiment the tumor is a gastric tumor/gastroesophageal junction tumor expressing CEACAM5. In a particular embodiment the tumor is a lung tumor. All therapeutic applications methods of use, uses, combinations, etc. described herein are especially embodiments for the treatment of these tumors/diseases.

The inventors recognize that the antibodies according to the invention show low or no anti-drug antibody (ADA) formation potential respectively loss of drug exposure due to neutralizing ADA respectively loss of efficacy given the IgG like structure and architecture.

In one embodiment, the invention provides a method of treating carcinomas (cancer, tumors, for example, human carcinomas), especially CEACAM5 expressing tumors, in vivo. This method comprises administering to a subject a pharmaceutically effective amount of a composition according to the invention. By “subject” is meant a human subject, in one embodiment a patient suffering from cancer/tumor/carcinoma.

CEACAM5 expression can be found in various tumor entities, especially in colorectal carcinoma, pancreatic adenocarcinoma, gastric cancer, non-small cell lung cancer, breast cancer among others. In healthy, normal glandular epithelia in the gastrointestinal tract, CEACAM5 is mainly expressed in a polarized pattern on the apical surface of the cells. This polarized expression pattern limits the accessibility by anti-CEA mono or bispecific antibodies which are administered alone systemically and therefore limits potential toxicity to healthy tissues. Administration of the second antibody together with the low affinity CD47 binding first antibody leads to no or limited killing/phagocytosis of such normal cells by the use according to the invention. This polarized expression pattern is no more present in cells of gastrointestinal- and other malignant tumors. CEACAM5 is expressed equally over the whole cell surface of cancer cells. This means that cancer cells are much better accessible to the first and second antibody than normal, healthy cells, and can be selectively killed by the combinations according to the invention. Expression of CEACAM5 in cancer cells is mostly higher than the expression in non-malignant cells.

In one embodiment, the combination according to the invention is used as a simultaneous, separate, or sequential combination. In one embodiment the combination is used in combination with a PD-1 axis antagonist in simultaneous, separate, or sequential combination. Such PD-1 axis antagonists are described e.g. in WO2017118675. Such combinations allow the attack of cancer cells in solid tumors by macrophages and T-cells.

As used herein the terms “combination, simultaneous, separate, or sequential combination” of the first antibody and the second antibody refer to any administration of the two antibodies (or three antibodies in case of the combination with a PD-1 axis antagonist), either separately or together, where the two or three antibodies are administered as part of an appropriate dose regimen designed to obtain the benefit of the combination therapy, for example in separate, sequential, simultaneous, concurrent, chronologically staggered or alternating administration. Thus, the two or three antibodies can be administered either as part of the same pharmaceutical composition or in separate pharmaceutical compositions. The first antibody can be administered prior to, at the same time as, or after the administration of the second bispecific antibody, or in some combination thereof. Where the first antibody is administered to the patient at repeated intervals the second bispecific antibody can be administered prior to, at the same time as, or after, each administration of the first antibody or some combination thereof, or at different intervals in relation to the treatment with the first antibody, or in a single dose prior to, at any time during, or subsequent to the course of treatment with the first antibody. In one embodiment, the first antibody and the second antibody are administered in alternating administration, in one embodiment, in intervals of 6 to 15 days between administration of the first antibody and the second antibody. In such alternating administration the first dose can be the first antibody or the second antibody.

The term “PD-1 axis antagonist” refers to an anti-PD-1 antibody or an anti-PD-L1 antibody. Anti-PD-1 antibodies are e.g. pembrolizumab (Keytruda®, MK-3475), nivolumab, pidilizumab, lambrolizumab, MEDI-0680, PDR001, and REGN2810. Anti-PD-1 antibodies are described e.g. in WO200815671, WO2013173223, WO2015026634, U.S. Pat. Nos. 7,521,051, 8,008,449, 8,354,509, WO20091 14335, WO2015026634, WO2008156712, WO2015026634, WO2003099196, WO2009101611, WO2010/027423, WO2010/027827, WO2010/027828, WO2008/156712, and WO2008/156712 (each of which is incorporated by reference in its entirety).

Anti-PD-L1 antibodies are e.g. atezolizumab, MDX-1 105, durvalumab and avelumab. Anti-PD-L1 antibodies are e.g. described in WO2015026634, WO2013/019906, WO2010077634, U.S. Pat. No. 8,383,796, WO2010077634, WO2007005874, and WO2016007235 (each of which is incorporated by reference in its entirety).

With regards to combined administration of the first and second antibody, both compounds may be present in one single dosage form or in separate dosage forms, for example in two different or identical dosage forms.

The first and second antibody are not competing regarding CEACAM5-binding and can therefore, if desired by the physician, be administered simultaneously.

The first and second antibody will typically be administered to the patient in a dose regimen that provides for the most appropriate treatment of the cancer both in terms of efficacy and safety), as known in the art. Preferably, tumor cells are attacked at the same time by T-cells and macrophages, to achieve full therapeutic potential of this approach. Therefore, CEA×CD3 and CEA×CD47 bispecific antibodies according to the invention have to be non-competitive regarding binding to CEA on cell surface.

As discussed above, the amount of the antibodies administered and the timing of the administration of the antibodies can depend on the type (e.g. gender, age, weight) and condition of the patient being treated, the severity of the disease or condition being treated, and on the route of administration. For example, the first and second antibody can be administered to a patient in doses ranging from 0.1 to 100 mg/kg of body weight per day or per week in single or divided doses, or by continuous infusion. In one embodiment, each of the antibodies is administered to a patient in doses ranging from 0.1 to 30 mg/kg. In some instances, dosage levels below the lower limit of the aforesaid range may be adequate, while in other cases still larger doses may be employed without causing any harmful side effect.

As used herein, the term “half-life of the antibody” refers to the elimination half-life of said antibody as measured in a usual pharmacokinetic assay. The first and second bispecific antibody have elimination half-life of 3-14 days.

In another aspect, the invention is also directed to the use of the combination according to the invention in the treatment of disease, particularly cell proliferation disorders wherein CEACAM5 is expressed, particularly wherein CEACAM5 is overexpressed (e.g., overexpressed or expressed in a different pattern on the cell surface) compared to normal tissue of the same cell type. Such disorders include, but are not limited to colorectal cancer, NSCLC (non-small cell lung cancer), gastric cancer, gastroesophageal cancer, pancreatic cancer, and breast cancer. CEACAM5 expression levels may be determined by different state-of-the-art methods (e.g., via immunohistochemistry assay, immunofluorescence assay, immunoenzyme assay, ELISA, flow cytometry, radioimmunoassay etc.).

In one aspect, the combination of the invention can be used for targeting cells in vivo or in vitro that express CEACAM5. The combination is particularly useful in eradication of tumors and inhibition of tumor growth or metastasis via the induction of ADCP and ADCC of tumor cells. The combination can be used to treat any tumor expressing CEACAM5. Particular malignancies that can be treated with the combination of the invention include, but are not limited to, colorectal cancer, non-small cell lung cancer, gastric cancer, gastroesophageal junction cancer, pancreatic cancer, and breast cancer.

The combination is administered to a human, in a pharmaceutically acceptable dosage form such as those discussed below, including those that may be administered to a human intravenously as a bolus or by continuous infusion over a period of time, by intramuscular, intraperitoneal, intra-cerebrospinal, subcutaneous, intra-articular, intrasynovial, intrathecal, topical, routes. The combination is also suitably administered by intra tumoral, peritumoral, intralesional, or perilesional routes, to exert local as well as systemic therapeutic effects.

For the treatment of disease, the appropriate dosage of the first and second antibody will depend on the type of disease to be treated, the severity and course of the disease, previous therapy, the patient's clinical history and response to the antibody, and the discretion of the attending physician. The first and second antibody are suitably administered to the patient at one time or over a series of treatments. The present invention provides a method for selectively killing tumor cells (also named herein as cancer cells) expressing CEACAM5.

This method comprises interaction of the first and second bispecific antibody with said tumor cells. These tumor cells may be from a human carcinoma including colorectal carcinoma, non-small cell lung carcinoma (NSCLC), gastric carcinoma, gastroesophageal junction cancer, pancreatic carcinoma and breast carcinoma.

In another aspect, the invention is directed to the use of the first and second antibody for the manufacture of a medicament for treating a disease related to CEACAM5 overexpression. In a particular embodiment, the disease is a cancer that expresses or even overexpresses CEACAM5, including but not limited to colorectal tumors, non-small cell lung tumors (NSCLC), gastric tumors, gastroesophageal junction tumors, pancreatic tumors, and breast tumors. In a particular embodiment, the tumors are colorectal tumors.

### Compositions, Formulations, Dosages, and Routes of Administration

In one aspect, the present invention is directed to pharmaceutical compositions comprising the first and second antibody and a pharmaceutically acceptable carrier. The present invention is further directed to the use of such pharmaceutical compositions in the method of treatment of disease, such as cancer, or in the manufacture of a medicament for the treatment of disease, such as cancer. Specifically, the present invention is directed to a method for the treatment of disease, and more particularly, for the treatment of cancer, the method comprising administering a therapeutically effective amount of the pharmaceutical composition of the invention.

In one aspect, the present invention encompasses pharmaceutical compositions, combinations, and methods for treating human carcinomas, tumors, as defined above. For example, the invention includes pharmaceutical compositions for use in the treatment of human carcinomas comprising a pharmaceutically effective amount of the first and second antibody and a pharmaceutically acceptable carrier.

The bispecific antibody composition of the invention can be administered using conventional modes of administration including, but not limited to, intravenous, intraperitoneal, oral, intralymphatic or direct intratumoral administration. Intravenous administration or subcutaneous administration are preferred.

In one aspect of the invention, therapeutic formulations containing the first and second antibody are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or liquid formulations. Acceptable carriers, excipients, or stabilizers are nontoxic to recipients at the dosages and concentrations employed. The formulations to be used for in vivo administration must be sterile. This is readily accomplished by filtration through sterile filtration membranes. The most effective mode of administration and dosage regimen for the pharmaceutical compositions of this invention depends upon the severity and course of the disease, the patient's condition and response to treatment and the judgment of the treating physician. Accordingly, the dosages of the compositions may be flat doses, or may be adapted to the individual patient, e.g. the body weight, or body weight per square meter body surface. Nevertheless, an effective dose of the compositions of this invention will generally be in a range from 0.1 to 30 mg/kg.

The first and second antibody have a molecular weight in a magnitude of 150 kDa per Mol. They carry in one embodiment a Fc part. The elimination half-life in patients is in a range of 3 to 14 days. This half-life allows for, but not limited to administration once a day, once a week, or once every two weeks or even 4 weeks.

The composition of the invention may be in a variety of dosage forms which include, but are not limited to, liquid solutions or suspensions, tablets, pills, powders, polymeric microcapsules or microvesicles, liposomes, and injectable or infusible solutions. The preferred form depends upon the mode of administration and the therapeutic application.

The composition will be formulated, dosed, and administered in a fashion consistent with good medical practice. Factors for consideration in this context include the particular disease or disorder being treated, the particular human being treated, the clinic condition of the individual patient, the cause of the disease or disorder, the site of delivery of the agent, the method of administration, the scheduling of administration, and other factors known to medical practitioners.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a bispecific antibody of the invention. The label or package insert indicates that the composition is used for treating the condition of choice. In one embodiment, the article of manufacture may comprise (a) a first container containing the first antibody, and a second container comprising the second antibody. In one embodiment, the article of manufacture may comprise both antibodies in one container. Moreover, the article of manufacture may comprise a further container comprising a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE 1
Sequence List

| Sequence | | |
|---|---|---|
| Number | Relates to | Sequence |
| SEQ ID NO: 1 | Common VH | EVQLVESGGGLVQPGGSLKLSCAASGFTENTYAMNWV |
| | | RQAPGKGLEWVGRIRSKYNNYATYYADSVKDRFTISR |
| | | DDSKNTAYLQMNSLKTEDTAVYYCVRHGNFGNSYVSW |
| | | FAYWGQGTLVTVSS |
| SEQ ID NO: 2 | Common CDRH1 | TYAMN |
| SEQ ID NO: 3 | Common CDRH2 | RIRSKYNNYATYYADSVKD |
| SEQ ID NO: 4 | Common CDRH3 | HGNFGNSYVSWFAY |
| SEQ ID NO: 5 | huCD3 VL 1A4 | QTVVTQEPSLTVSPGGTVTLTCRSSTGAVTTSNYANW |
| | | FQQKPGQAPRGLIGGTNKRAPGTPARFSGSLLGGKAA |
| | | LTLSGAQPEDEAEYYCALWYKQRWVFGGGTKLTVL |
| SEQ ID NO: 6 | huCD3 1A4 | RSSTGAVTTSNYAN |
| | CDRL1 | |
| SEQ ID NO: 7 | huCD3 1A4 | GTNKRAP |
| | CDRL2 | |
| SEQ ID NO: 8 | huCD3 1A4 | ALWYKQRWV |
| | CDRL3 | |
| SEQ ID NO: 9 | huCD3 1A4 LC | QTVVTQEPSLTVSPGGTVTLTCRSSTGAVTTSNYANW |
| | | FQQKPGQAPRGLIGGTNKRAPGTPARFSGSLLGGKAA |
| | | LTLSGAQPEDEAEYYCALWYKQRWVFGGGTKLTVLGQ |
| | | PKAAPSVTLFPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTECS |
| SEQ ID NO: 10 | Common constant | ASTKGPSVFPLAPSSKSTSGGTAALGCLVKDYFPEPV |
| | heavy chain (WT | TVSWNSGALTSGVHTFPAVLQSSGLYSLSSVVTVPSS |
| | IgG1) | SLGTQTYICNVNHKPSNTKVDKRVEPKSCDKTHTCPP |
| | | CPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPG |
| SEQ ID NO: 11 | kappa light chain | FGQGTKVEIKRTVAAPSVFIFPPSDEQLKSGTASVVC |
| | constant region | LLNNFYPREAKVQWKVDNALQSGNSQESVTEQDSKDS |
| | (CK) | |
| | | TYSLSSTLTLSKADYEKHKVYACEVTHQGLSSPVTKS |
| | | ENRGEC |
| SEQ ID NO: 12 | lambda light | FGGGTKLTVLGQPKAAPSVTLFPPSSEELQANKATLV |
| | chain constant | CLISDFYPGAVTVAWKADSSPVKAGVETTTPSKQSNN |
| | region (CL) | KYAASSYLSLTPEQWKSHRSYSCQVTHEGSTVEKTVA |
| | | PTECS |
| SEQ ID NO: 13 | Common heavy | EVQLVESGGGLVQPGGSLKLSCAASGFTENTYAMNWV |
| | chain (wild-type) | RQAPGKGLEWVGRIRSKYNNYATYYADSVKDRFTISR |
| | | DDSKNTAYLQMNSLKTEDTAVYYCVRHGNFGNSYVSW |
| | | FAYWGQGTLVTVSSASTKGPSVFPLAPSSKSTSGGTA |
| | | ALGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPELLGGPSVFLFPPKPKDTL |
| | | MISRTPEVTCVVVDVSHEDPEVKFNWYVDGVEVHNAK |
| | | TKPREEQYNSTYRVVSVLTVLHQDWLNGKEYKCKVSN |
| | | KALPAPIEKTISKAKGQPREPQVYTLPPSREEMTKNQ |
| | | VSLTCLVKGFYPSDIAVEWESNGQPENNYKTTPPVLD |
| | | SDGSFFLYSKLTVDKSRWQQGNVFSCSVMHEALHNHY |
| | | TQKSLSLSPG |
| SEQ ID NO: 14 | Common heavy | EVQLVESGGGLVQPGGSLKLSCAASGETENTYAMNWV |
| | chain (LALA | RQAPGKGLEWVGRIRSKYNNYATYYADSVKDRFTISR |
| | mutation) | DDSKNTAYLQMNSLKTEDTAVYYCVRHGNEGNSYVSW |
| | | FAYWGQGTLVTVSSASTKGPSVFPLAPSSKSTSGGTA |
| | | ALGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAAGGPSVFLFPPKPKDTL |
| | | MISRTPEVTCVVVDVSHEDPEVKENWYVDGVEVHNAK |
| | | TKPREEQYNSTYRVVSVLTVLHQDWLNGKEYKCKVSN |
| | | KALPAPIEKTISKAKGQPREPQVYTLPPSREEMTKNQ |
| | | VSLTCLVKGFYPSDIAVEWESNGQPENNYKTTPPVLD |
| | | SDGSFFLYSKLTVDKSRWQQGNVFSCSVMHEALHNHY |
| | | TQKSLSLSPG |
| SEQ ID NO: 15 | Common heavy | EVQLVESGGGLVQPGGSLKLSCAASGFTENTYAMNWV |
| | chain | RQAPGKGLEWVGRIRSKYNNYATYYADSVKDRETISR |
| | (LALA + P329A | DDSKNTAYLQMNSLKTEDTAVYYCVRHGNFGNSYVSW |
| | mutation) | FAYWGQGTLVTVSSASTKGPSVFPLAPSSKSTSGGTA |
| | | ALGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPEAAGGPSVELFPPKPKDTL |
| | | MISRTPEVTCVVVDVSHEDPEVKFNWYVDGVEVHNAK |
| | | TKPREEQYNSTYRVVSVLTVLHQDWLNGKEYKCKVSN |
| | | KALAAPIEKTISKAKGQPREPQVYTLPPSREEMTKNQ |
| | | VSLTCLVKGFYPSDIAVEWESNGQPENNYKTTPPVLD |
| | | SDGSFFLYSKLTVDKSRWQQGNVFSCSVMHEALHNHY |
| | | TQKSLSLSPG |
| SEQ ID NO: 16 | hybrid-kappa | FGGGTKLTVLGRTVAAPSVFIFPPSDEQLKSGTASVV |
| | light chain | CLLNNFYPREAKVQWKVDNALQSGNSQESVTEQDSKD |
| | constant region | STYSLSSTLTLSKADYEKHKVYACEVTHQGLSSPVTK |
| | (H-CK 5) | SENRGEC |
| SEQ ID NO: 17 | huCD3 1A4 LC- | QTVVTQEPSLTVSPGGTVTLTCRSSTGAVTTSNYANW |
| | hybrid kappa | FQQKPGQAPRGLIGGTNKRAPGTPARFSGSLLGGKAA |
| | | LTLSGAQPEDEAEYYCALWYKQRWVFGGGTKLTVLGR |
| | | TVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSENRGEC |
| SEQ ID NO: 18 | CEA AB73 | RASQSVNSNLN |
| | CDRL1 | |
| SEQ ID NO: 19 | CEA AB73 | HSNNRPH |
| | CDRL2 | |
| SEQ ID NO: 20 | CEA AB73 | QQFDYFREYNT |
| | CDRL3 | |
| SEQ ID NO: 21 | CEA AB73 VL | EIVLTQSPATLSLSPGERATLSCRASQSVNSNLNWYQ |
| | light chain | QKPGQAPRLLIYHSNNRPHGIPARFSGSGSGTDFTLT |
| | variable region | ISSLEPEDFAVYYCQQFDYFREYNTFGQGTKVEIK |
| SEQ ID NO: 22 | CEA AB73 LC | EIVLTQSPATLSLSPGERATLSCRASQSVNSNLNWYQ |
| | light chain | QKPGQAPRLLIYHSNNRPHGIPARFSGSGSGTDETLT |
| | | ISSLEPEDFAVYYCQQFDYFREYNTFGQGTKVEIKRT |
| | | VAAPSVFIFPPSDEQLKSGTASVVCLLNNFYPREAKV |
| | | QWKVDNALQSGNSQESVTEQDSKDSTYSLSSTLTLSK |
| | | ADYEKHKVYACEVTHQGLSSPVTKSENRGEC |
| SEQ ID NO: 23 | Common heavy | SYAMS |
| | chain CDRH1 | |
| SEQ ID NO: 24 | Common heavy | AISGSGGSTYYADSVKG |
| | chain CDRH2 | |
| SEQ ID NO: 25 | Common heavy | SYGAFDY |
| | chain CDRH3 | |
| SEQ ID NO: 26 | Common heavy | EVQLLESGGGLVQPGGSLRLSCAASGFTFSSYAMSWV |
| | chain variable | RQAPGKGLEWVSAISGSGGSTYYADSVKGRFTISRDN |
| | region VH | SKNTLYLQMNSLRAEDTAVYYCAKSYGAFDYWGQGTL |
| | | VTVSS |
| SEQ ID NO: 27 | Common heavy | EVQLLESGGGLVQPGGSLRLSCAASGFTFSSYAMSWV |
| | chain (VH-CH1) | RQAPGKGLEWVSAISGSGGSTYYADSVKGRFTISRDN |
| | | SKNTLYLQMNSLRAEDTAVYYCAKSYGAFDYWGQGTL |
| | | VTVSSASTKGPSVFPLAPSSKSTSGGTAALGCLVKDY |
| | | FPEPVTVSWNSGALTSGVHTFPAVLQSSGLYSLSSVV |
| | | TVPSSSLGTQTYICNVNHKPSNTKVDKRVEPKSCDKT |
| | | HTCPPCP |
| SEQ ID NO: 28 | common heavy | EVQLLESGGGLVQPGGSLRLSCAASGFTESSYAMSWV |
| | chain (VH-CH1- | RQAPGKGLEWVSAISGSGGSTYYADSVKGRFTISRDN |
| | CH2-CH3) | SKNTLYLQMNSLRAEDTAVYYCAKSYGAFDYWGQGTL |
| | | VTVSSASTKGPSVFPLAPSSKSTSGGTAALGCLVKDY |
| | | FPEPVTVSWNSGALTSGVHTFPAVLQSSGLYSLSSVV |
| | | TVPSSSLGTQTYICNVNHKPSNTKVDKRVEPKSCDKT |
| | | HTCPPCPAPELLGGPSVELFPPKPKDTLMISRTPEVT |
| | | CVVVDVSHEDPEVKENWYVDGVEVHNAKTKPREEQYN |
| | | STYRVVSVLTVLHQDWLNGKEYKCKVSNKALPAPIEK |
| | | TISKAKGQPREPQVYTLPPSREEMTKNQVSLTCLVKG |
| | | FYPSDIAVEWESNGQPENNYKTTPPVLDSDGSFFLYS |
| | | KLTVDKSRWQQGNVFSCSVMHEALHNHYTQKSLSLSP |
| | | G |
| SEQ ID NO: 29 | CD47 binding | RASQSISSYLN |
| | part CDRLI | |
| SEQ ID NO: 30 | CD47 binding | AASSLQS |
| | part CDRL2 | |
| SEQ ID NO: 31 | CD47 binding | QQMHPRAPKT |
| | part CDRL3 | |
| SEQ ID NO: 32 | CD47 binding | DIQMTQSPSSLSASVGDRVTITCRASQSISSYLNWYQ |
| | part light chain | QKPGKAPKLLIYAASSLQSGVPSRFSGSGSGTDETLT |
| | variable region | ISSLQPEDFATYYCQQMHPRAPKTFGQGTKVEIK |
| | VK | |
| SEQ ID NO: 33 | CD47 binding | DIQMTQSPSSLSASVGDRVTITCRASQSISSYLNWYQ |
| | part light chain | QKPGKAPKLLIYAASSLQSGVPSRESGSGSGTDFTLT |
| | (VKCK; K2) | ISSLQPEDFATYYCQQMHPRAPKTFGQGTKVEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSENRGEC |
| SEQ ID NO: 34 | CD47 binding | gacatccaga tgacccagtc tccatcctcc |
| | part light chain | ctgtctgcat ctgtaggaga cagagtcacc |
| | (VKCK; nucleic | atcacttgcc gggcaagtca gagcattagc |
| | acid); (K2) | agctatttaa attggtatca gcagaaacca |
| | | gggaaagccc ctaagctcct gatctatgct |
| | | gcatccagtt tgcaaagtgg ggtcccatca |
| | | aggttcagtg gcagtggatc tgggacagat |
| | | ttcactctca ccatcagcag tctgcaacct |
| | | gaagattttg caacttacta ctgtcagcag |
| | | atgcacccgc gcgccccgaa gaccttcggc |
| | | caagggacca aggtggaaat caaacgtacg |
| | | gtggctgcac catctgtctt catcttcccg |
| | | ccatctgatg agcagttgaa atctggaact |
| | | gcctctgttg tgtgcctgct gaataacttc |
| | | tatcccagag aggccaaagt acagtggaag |
| | | gtggataacg ccctccaatc gggtaactcc |
| | | caggagagtg tcacagagca ggacagcaag |
| | | gacagcacct acagcctcag cagcaccctg |
| | | acgctgagca aagcagacta cgagaaacac |
| | | aaagtctacg cctgcgaagt cacccatcag |
| | | ggcctgagct cgcccgtcac aaagagcttc |
| | | aacaggggag agtgttaa |
| SEQ ID NO: 35 | CEACAM5 | SGSSSNIGYGLVS |
| | binding part | |
| | AC100 CDRL1 | |
| SEQ ID NO: 36 | CEACAM5 | NGNIRPS |
| | binding part | |
| | AC100 CDRL2 | |
| SEQ ID NO: 37 | CEACAM5 | GTWDFSYRVD |
| | binding part | |
| | AC100 CDRL3 | |
| SEQ ID NO: 38 | CEACAM5 | QSVLTQPPSVSAAPGQKVTISCSGSSSNIGYGLVSWY |
| | binding part light | QQLPGTAPKLLIYNGNIRPSGIPDRESGSKSGTSATL |
| | chain variable | GITGLQTGDEADYYCGTWDFSYRVDFGGGTKLTVL |
| | region AC100 VL | |
| SEQ ID NO: 39 | CEACAM5 | QSVLTQPPSVSAAPGQKVTISCSGSSSNIGYGLVSWY |
| | binding part | QQLPGTAPKLLIYNGNIRPSGIPDRFSGSKSGTSATL |
| | AC100 light chain | GITGLQTGDEADYYCGTWDFSYRVDFGGGTKLTVLGQ |
| | VLCL | PKAAPSVTLEPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTECS |
| SEQ ID NO: 40 | Primer | TTGTGTGACTCTTAACTCTCAGAG |
| SEQ ID NO: 41 | Primer | GAGGCCACTTGTGTAGCGCCAAGTG |
| SEQ ID NO: 42 | Primer | GTGAGTCCATGGCTGTCACTG |
| SEQ ID NO: 43 | Primer | CCTGACTTGGCTATTCTCAG |
| SEQ ID NO: 44 | Primer | GTCTGAAGCATTATGTGTTGAAGC |
| SEQ ID NO: 45 | Primer | GTGAGTACATTCATTGTACTGTG |

Sequences No. 1 to 23 refer to antibody AB73 and sequences 24 to 39 refer to K2AC100.

### EXAMPLES

### Example 1: Cloning, Expression and Purification of Human CEACAM5; Source of huCEACAM3 and huCD47

The sequence corresponding to the complete extracellular domain (ECD) CEACAM5 were subcloned into the pEAK8 mammalian expression vector (Edge Biosystems, Gaithersburg, Md.).

The vectors were modified to introduce an Avitag™ (Avidity, Denver Colo.) and a hexa-histidine tag, a human Fc region or a mouse Fc region at the C-terminus. Constructs were verified by DNA sequencing. Purification of recombinant soluble protein was carried out by IMAC (Immobilized Metal Ion Affinity Chromatography), FcXL or CaptureSelect™ IgG-Fc (ms) Affinity Matrix; Human CEACAM3 and biotinylated CEACAM3 are available from ACROBiosystems, Newark USA (Thermo Fisher Scientific). Human CD47 and biotinylated CD47 can be produced as described in WO2019234576 or are available from ACROBiosystems, Newark USA.

### Example 2: Expression and Purification of Bispecific Antibodies Carrying a Lambda and a Kappa Light Chain

Simultaneous expression can be achieved in different ways such as the transfection of multiple vectors, each expressing one of the chains to be co-expressed, or by using vectors that drive expression of multiple genes. A vector pNovi κHλ was previously generated to allow for the co-expression of one heavy chain, one Kappa light chain and one Lambda light chain as described in US 2012/0184716 and WO 2012/023053, each of which is hereby incorporated by reference in its entirety. The expression of the three genes is driven by human cytomegalovirus promoters (hCMV) and the vector also contains a glutamine synthetase gene (GS) that enables the selection and establishment of stable cell lines. The VL genes of the anti-hCEACAM5 IgGλ or the anti-hCD47 IgGκ were cloned in the vector pNovi κHλ, for transient expression in mammalian cells. Peak cells or CHO cells are cultured in appropriate Flask with suitable cells number and culture medium volume (containing fetal bovine serum). Plasmid DNA is transfected into the cells using Lipofectamine 2000) according to manufacturer's instructions. Antibody concentration in the supernatant of transfected cells is measured during the production using OctetRED96. According to antibody concentration, supernatants are harvested 5 to 7 days after transfection and clarified by centrifugation at 1300 g for 10 min. The purification process is composed of three affinity steps. First, the FcXL affinity matrix (Thermo Fisher Scientific) is washed with PBS and then added in the clarified supernatant. After incubation overnight at +4° C., supernatants are centrifuged at 2000 g for 10 min, flow through is stored and resin washed twice with PBS. Then, the resin is transferred on Amicon™ Pro columns and a solution containing 50 mM glycine at pH 3.0 is used for elution. Several elution fractions are generated, pooled and desalted against PBS using 50 kDa Amicon™ Ultra Centrifugal filter units (Merck KGaA, Darmstadt, Germany). The eluted product, containing total human IgGs from the supernatant, is quantified using a Nanodrop spectrophotometer (NanoDrop Technologies, Wilmington, Del.) and incubated for 15 min at RT and 20 rpm with the appropriate volume of Kappa select affinity matrix (GE Healthcare). Incubation, resin recovery, elution and desalting steps are performed as described previously. The last affinity purification step is performed using the lambda Fab select affinity matrix (GE Healthcare) applying the same process as for the two previous purifications. The final product is quantified using the Nanodrop. Purified bispecific antibodies are analyzed by electrophoresis in denaturing and reducing conditions. The Agilent 2100 Bioanalyzer is used with the Protein 80 kit as described by the manufacturer (Agilent Technologies, Santa Clara, Calif, USA). 4 μL of purified samples are mixed with sample buffer supplemented with dithiothreitol (DTT; Sigma Aldrich, St. Louis, Mo.). Samples are heated at 95° C. for 5 min and then loaded on the chip. All samples are tested for endotoxin contamination using the Limulus Amebocyte Lysate test (LAL; Charles River Laboratories, Wilmington, Mass., USA).

### Example 3: KD Measurement

### a) Experimental Procedure to Measure the KD of an Ab to Recombinant Human CEACAM5 (Octet)

The affinity of the anti-human CEACAM5 arm of the CD47×CEACAM5 bispecific antibodies of the invention for recombinant soluble human CEACAM5 was determined using the Bio-Layer Interferometry (BLI) technology. An OctetRED96 instrument and Protein A biosensors were used (Sartorius). The measurement was performed at 30° C. After hydration, pre-conditioning, and a baseline step in Kinetic buffer (PBS, 0.002% Tween 20, 0.01% BSA, Kathon; Sartorius), biosensors were loaded for 5 min with the d, body at 0.5 μg/mL in Kinetic buffer. Then, biosensors were dipped into a serial dilution of recombinant human CEACAM5 Extra Cellular Domain (ECD) soluble protein (produced in house), starting at 50 nM with a 2× dilution factor. The association and dissociation phases were monitored for 600 seconds each. Biosensors were regenerated using 10 mM glycine pH 1.7. A standard acquisition rate was applied (5.0 Hz, averaging by 20). Curves were processed with a reference well subtraction, a Y alignment on the baseline, without interstep correction. The affinity was measured applying a 1:1 global fitting model on the full association and dissociation steps. The binding affinity (KD) of the bispecific antibodies of the invention to recombinant human CD47 was determined by the same experimental procedure. The KD's of exemplary bispecific antibodies of the invention to CEACAM5, as determined by this procedure, are shown in Table 2 below.

### b) Experimental Procedure to Measure the KD of an Ab to Recombinant Human CEACAM3 (Octet)

The affinity of the anti-human CEACAM5 arms of the CD47×CEACAM5 bispecific antibodies of the invention for recombinant soluble human CEACAM3 was determined using the Bio-Layer Interferometry (BLI) technology and an OctetRED96 instrument. HIS1K biosensors (Sartorius), loaded with an anti-His tag antibody, were used to capture his-tagged recombinant huCEACAM3 (R&D Systems, #9868-CM). The measurement was performed at 30° C. After hydration, pre-conditioning and a baseline step in Kinetic buffer (PBS, 0.002% Tween 20, 0.01% BSA, Kathon; Sartorius), biosensors were loaded for 5 min with the recombinant huCEACAM3 at 5 μg/mL in Kinetic buffer. Then, biosensors were dipped into a serial dilution of d, bodies, starting at 667 nM with a 2× dilution factor. The association and dissociation phases were monitored for 60 seconds and 120 seconds respectively. Biosensors were regenerated using 10 mM glycine pH 1.7. A standard acquisition rate was applied (5.0 Hz, averaging by 20). Curves were processed with a double reference subtraction, a Y alignment on the baseline and an interstep correction. The affinity was measured applying a 1:1 global fitting model on the full association step and the first 5 seconds of the dissociation step. The KD's of exemplary bispecific antibodies of the invention to CEACAM3, as determined by this procedure, are shown in Table 2 below.

TABLE 2
Binding affinities (KD; nM) of the anti-CEACAM5 arm
for the CEACAM5 × CD47 bispecific antibodies
K2AC100 and K2AC22 (comparison) measured by Octet.

| | Affinity to rec. | | Affinity to rec. | | Fold difference |
|---|---|---|---|---|---|
| | hCEACAM5 | | hCEACAM3 | | hCEACAM3/ |
| | (KD; nM) | | (KD; nM) | | hCEACAM5 |
| K2AC22 | 120 | (+/−13) | 10′000 | (+/−3000) | 83 |
| K2AC100 | 4.8 | (+/−1) | 700 | (+/−270) | 146 |

### Example 4: Epitope Binning of CEACAM5×CD47 Bispecific Antibodies by Competition with Reference Antibody SM3E

Epitope binning is a competitive immunoassay used to characterize the binding of antibodies according to the invention or, e.g., the binding of the related bivalent anti-CEA (target protein) antibodies of the first binding part of the bispecific antibodies of the invention. A competitive blocking profile of a new antibody binding to the target protein is created against antibodies also binding to this target protein and for which the binding epitope has already been established/published. Competition to this reference antibody indicate that the antibody has the same or a topographically closely related epitope, so that they are “binned” together. The ability of the CD47×CEACAM5 bispecific antibodies of the present invention to compete with CEACAM5 reference antibodies is tested by ELISA on recombinant human CEACAM5 with reference antibody derived from SM3E (US20050147614) carrying a mouse Fc region (mAb produced using standard methods). SM3E binds more to the N-terminal, cell membrane distal part of CEA.

Biotinylated human CEACAM5 is coated at 0.5 g/ml in a Streptavidin-coated 96-well plate and incubated with serial dilutions of the reference mAb (from 0.09 nM to 67 nM) or an unrelated mAb carrying a mouse Fc region for 1 hour. The CD47×CEACAM5 bispecific antibodies of the present invention are added at 0.1 g/ml for 1 hour at room temperature. The plate is washed and the bound CD47×CEACAM5 bispecific antibodies are detected with an anti-human IgG(Fc)-HRP (Jackson ImmunoResearch). After washing, the plate is revealed with Amplex Red reagent. The fluorescence signal is measured on a Synergy HT plate reader (Biotek).

Competition experiments were performed with the CD47×CEACAM5 bispecific antibodies of the present invention. Binding of K2AC100 was reduced by the respective competitive (i.e., tool) antibody by 80% or more. A CD47×CEACAM5 bispecific antibody is identified herein as competitive with SM3E antibody when binding of the bispecific antibody is reduced by 80% or more with the highest concentration of the reference tool antibody. A CD47×CEACAM5 bispecific antibody is identified as non-competitive with a tool antibody in case binding to CEACAM5 is reduced by less than 20% if the results with and w/o addition of a tool antibody are compared.

### Example 5: Quantification of the Target Density (i.e. Number of Molecules) of CEACAM5 and CD47 on the Cell Surface of Six Different Cancer Cell Lines

These cell lines are human gastric adenocarcinoma cells (MKN-45, DSMZ ACC 409), human colorectal cancer cells (SK-CO-1 (ATCC; HTB-39); SNU-C1 (ATCC; CRL-5972); Ls174T (ATCC; CL-188) and LoVo (ATCC; CCL-229)), or pancreatic adenocarcinoma cells (HPAF-II, ATCC, CRL-1997).

QIFIKIT® (Agilent Dako) is used for the quantitative determination of cell surface antigens by flow cytometry using an indirect immunofluorescence assay. QIFIKIT® consists of a series of 6 bead populations coated with different, but well-defined quantities of a mouse monoclonal antibody (Mab). The beads mimic cells labeled with a specific primary mouse monoclonal antibody. Different cell specimens may be labeled with different primary antibodies and then quantitated using the same set of calibration beads.

Cells were cultured in their adapted medium, detached with trypsin-EDTA (Sigma Aldrich), centrifuged (3 min, 350 g) and resuspended in cold FACS buffer (PBS, 2% BSA—from Sigma Aldrich), filtered through 0.22 μm (Stericup, Millipore)) to obtain 3.10⁵ cells/mL. 3.10⁵ cells of each sample were plated in a V-bottom plate. One L of FcγR blocking reagent was added to each well and the plate incubated at 4° C. for 10 min. Ten L of primary antibody against human CEACAM5 (#sc-23928; mIgG1 (Santa Cruz)) and human CD47 (internal production; B6H12; mouse backbone), at a final concentration 20 g/mL, were added to the cells and incubated 30 min at 4° C. Cells were washed twice with 200 μL of PBS BSA 2% and centrifuged at 400 g for 3 min. 100 μL of beads (Setup or Calibration from QIFIKIT®) were washed along with the cells and treated identically. One hundred μL of secondary antibody from the kit (1/50 in PBS BSA 2%) were added to each well and incubated for 30 to 45 min at 4° C. Cells were centrifuged (3 min, 400 g at 4° C.) to discard the supernatant and washed twice. After the last centrifugation, cells were resuspended in 130 μL of CellFix and acquired on CytoFlex cytometer (Beckman Coulter). Analysis was done using Flowlo software and Geometric means exported to an Excel file. A linear regression was performed using MFI values from the calibration beads. Antibody Binding Capacity (ABC) of cells were extrapolated from this regression line. Specific Antibody Binding Capacity (sABC) was obtained by subtracting ABC from isotype control to the one of the specific staining. Data from this analysis is presented in Table 3 below.

TABLE 3
Target density of CEACAM5 and CD47 at the
cell surface of six cancer cell lines.

| | Origin | CEACAM5 (×10³) | CD47 (×10³) |
|---|---|---|---|
| SK-CO-1 | Colorectal | 257 | 105 |
| MKN-45 | Gastric | 155 | 135 |
| HPAF-II | Pancreas | 120 | 114 |
| SNU-C1 | Colorectal | 85 | 68 |
| Ls174T | Colorectal | 26 | 57 |
| LoVo | Colorectal | 4 | 25 |

### Example 6: Measurement of the Binding of CEA×CD47 Bispecific Antibodies to CEACAM5 Expressing Cancer Cell Lines (EC50 and Maximal Binding (Emax))

The binding of CD47×CEACAM5 bispecific antibodies can be tested on CEACAM5-expressing human gastric adenocarcinoma cells (e.g., MKN-45), on CEACAM5-expressing human colorectal cancer cells (SK-CO-1, SNU-C1, Ls174T, and LoVo), and on CEACAM5-expressing pancreatic adenocarcinoma cells (HPAF-II).

Cells are harvested, counted, checked for viability, and resuspended at 3×10⁶ cells/ml in FACS buffer (PBS 2% BSA, 0.1% NaN₃). 100 μl of the cell suspension are distributed in V-bottom 96-well plates (3×10⁵ cells/well). The supernatant is removed by centrifugation 3 minutes at 4° C., 1300 rpm. Increasing concentrations of the antibody according to the invention are then added into the wells and incubated for 15 minutes at 4° C. Cells are washed twice with cold FACS buffer and re-incubated for further 15 minutes at 4° C. with the PE (R-phycoerythrin)-conjugated mouse anti-human IgG Fc secondary antibody (SouthernBiotech, pre-diluted 1:100 in FACS buffer). Cells are washed twice with cold FACS buffer and resuspended in 300 μl FACS buffer with 1:15000-diluted SytoxBlue (Life Technologies). Fluorescence respectively mean fluorescence activity (MFI) is determined using a Cytoflex (Millipore) flow cytometer. Binding curves and EC50 and Emax values are obtained and calculated using GraphPad Prism7 software. Data from this analysis is presented in Table 4 below.

TABLE 4
EC50 (nM) and Eₘₐₓ (MFI) binding of K2AC100 and K2AC22
to human cancer cell lines expressing CEACAM5 and CD47.

CD47 × CEACAM5
bispecific antibodies

| | | |
|---|---|---|
| Cell lines | K2AC22 | K2AC100 |

| | | | |
|---|---|---|---|
| SK- | EC50 (nM) | 24 | 14.7 |
| CO-1 | Emax | 0.52 | 0.9 |
| | (MFI*; ×10⁶) | | |
| MKN- | EC50 (nM) | 21.3 | 8.7 |
| 45 | Emax | 1.16 | 1.3 |
| | (MFI*; ×10⁶) | | |
| HPAF- | EC50 (nM) | 19.3 | 8 |
| II | Emax | 0.67 | 1.1 |
| | (MFI*; ×10⁶) | | |
| SNU- | EC50 (nM) | 10.7 | 3.27 |
| C1 | Emax | 0.24 | 0.3 |
| | (MFI*; ×10⁶) | | |
| Ls174T | EC50 (nM) | 28 | 4.5 |
| | Emax | 0.073 | 0.07 |
| | (MFI*; ×10⁶) | | |
| LoVo | EC50 (nM) | 44 | 18.7 |
| | Emax | 0.23 | 0.28 |
| | (MFI*; ×10⁶) | | |

*MFI—Mean Fluorescence Intensity
N/A—Not Applicable - no available data for this Ab on this cell line
K2AC100 shows a considerably lower EC50 value and a higher Emax value compared to K2AC22.
K2AC100 binds to SK-CO1 cells with an EC50 value of 10 to 30 nM, to MKN-45 cells with an EC50 value of 5 to 15 nM, to HPAF-II cells with an EC50 value of 5 to 15 nM, to SNU-C1 cells with an EC50 value of 1 to 10, to LS174T cells with an EC50 value of 3 to 15 nM, and/or to LoVo cells with an EC50 value of 15 to 25 nM.
K2AC100 binds to SK-CO1 cells with an Emax value of 0.5 to 1.5 (MFI × 10⁶), to MKN-45 cells with an Emax value of 1 to 2 (MFI × 10⁶), to HPAF-II cells with an Emax value of 0.5 to 1.5 (MFI × 10⁶), to SNU-C1 cells with an Emax value of 0.2 to 0.6 (MFI × 10⁶), to LS174T cells with an Emax value of 0.05 to 0.2 (MFI × 10⁶), and/or to LoVo cells with an Emax value of 0.2 to 0.5 (MFI × 10⁶).

### Example 7: Measurement of Phagocytosis (Phagocytosis Index) of Antibody Dependent Cellular Phagocytosis (ADCP), Respectively

The phagocytic in vitro activity of K2AC100 is assessed using 6 CEACAM5-expressing cancer cell lines (MKN-45, SK-CO-1, SNU-C1, Ls174T, LoVo, and HPAF-II). K2AC22 is assessed, for comparison, using the same cell lines and experimental procedures.

The assay relies on an imaging-based method, which makes use of the CellInsight CX5 High Content Screening Platform. The assessed readout is the phagocytosis index, defined as the average number of target cells engulfed by 100 macrophages.

### 1. Preparation of the Macrophages:

Human peripheral blood mononuclear cells (PBMCs) are isolated from buffy coats from different healthy donors, (from 5 to 7 different donors, depending on the cell line), by Ficoll gradient centrifugation. Macrophages are generated by culturing PBMCs for 7 to 9 days in complete medium (RPMI 1640, 10% heat-inactivated fetal calf serum (Invitrogen)), 2 mM L-glutamine, 1 mM sodium pyruvate, 10 mM HEPES buffer, 25 mg/mL gentamicin (all from Sigma-Aldrich), and 50 mM 2-mercaptoethanol (Thermo Fisher Scientific) in the presence of 20 ng/mL of human macrophage colony-stimulating factor (M-CSF, PeproTech). Non-adherent cells are subsequently eliminated in the differentiation phase (day +1) by exchanging the cell culture medium, and adherent cells representing macrophages are detached using cell dissociation buffer (Sigma-Aldrich) and washed in complete medium the day of use (day +7, day +8, or day +9) for ADCP experiment based on cytometry. For ADCP based on cell imaging, macrophages are detached at day +6 using cell dissociation buffer and seeded at 30′000 per well in 96 optical plate (Costar).

### 2. Assessment of the Phagocytosis Activity (CellInsight™ Based Assay)

Macrophages (stained with calcein red orange) adhering to microplate wells are co-incubated with Calcein AM-labeled target tumor cells at an effector:target cells ratio of 1:3 for 30 min (MKN45 and SNU-C1) or 2.5 hours (LoVo and Ls174T) at 37 degree C. in the presence of different concentrations of the tested antibodies. At the end of the incubation period, supernatants are replaced by complete culture medium and the microplates are imaged with the CellInsight™ CX5 High Content Screening Platform. 1500 macrophages are acquired and analyzed per well. Phagocytosis is evidenced as double-positive events (macrophage+target tumor cell) and the phagocytosis indexes are calculated by the CellInsight™ manufacturer's software.

All the results shown in FIG. 2 and Tables 5, 6, 7, 8, 9 are obtained with 4 CEACAM5-expressing cancer cell lines (MKN-45, SNU-C1, Ls174T, LoVo); with an effector cell to target/tumor cell ratio of 1.3.

TABLE 5
Percentage of increase in the maximum of phagocytosis
index assessed for K2AC100 in relation to K2AC22.

| | CEACAM5 | |
|---|---|---|
| | levels | K2AC100 |
| MKN-45 | 155′000 | 1.8 |
| SNU-C1 | 85′000 | 0 |
| Ls174T | 26′000 | 14.4 |
| LoVo | 4′000 | 18.6 |

K2AC100 shows better binding compared to K2AC22 (lower EC50 and higher Emax, see Example 6, Table 4). Surprisingly, the % increase of the maximal achieved phagocytosis index Emax ADCP of K2AC100 compared to K2AC22 is strongest in the low CEACAM5 expressing cell lines LoVo and Ls174T.

These results are obtained in experiments using macrophages obtained from different human donors. The data obtained from such experiments are shown in Table 6 (for MKN-45 cells), Table 7 (for SNU-C1 cells), in Table 8 (for Ls174T cells) and Table 9 (for LoVo cells).

TABLE 6
In vitro assessment of EC50 (μg/mL) and Eₘₐₓ for phagocytosis
activity of CEACAM5 × CD47 bispecific antibodies K2AC100
and K2AC22 (comparison)) using MKN-45 human cancer cell line
as target with 7 different donors (D) of macrophages.

| | | CD47 × CEACAM5 | | | |
|---|---|---|---|---|---|
| | | bispecific antibodies | | | |
| Donors (D) | | K2AC22 | | K2AC100 | |
| D854 | EC50 | 0.2 | | 0.3 | |
| | (μg/mL) | | | | |
| | Max index of | 57 | | 53 | |
| | phagocytosis | | | | |
| D860 | EC50 | 0.2 | | 0.25 | |
| | (μg/mL) | | | | |
| | Max index of | 41 | | 47 | |
| | phagocytosis | | | | |
| D864 | EC50 | 0.82 | | 0.53 | |
| | (μg/mL) | | | | |
| | Max index of | 54 | | 67 | |
| | phagocytosis | | | | |
| D867 | EC50 | 0.5 | | 0.21 | |
| | (μg/mL) | | | | |
| | Max index of | 62 | | 60 | |
| | phagocytosis | | | | |
| D868 | EC50 | 0.65 | | 0.16 | |
| | (μg/mL) | | | | |
| | Max index of | 31 | | 33 | |
| | phagocytosis | | | | |
| D870 | EC50 | 0.35 | | 0.1 | |
| | (μg/mL) | | | | |
| | Max index of | 40 | | 32 | |
| | phagocytosis | | | | |
| D871 | EC50 | 0.4 | | 0.2 | |
| | (μg/mL) | | | | |
| | Max index of | 41 | | 40 | |
| | phagocytosis | | | | |
| Mean | EC50 | 0.45 | (+/−0.2) | 0.25 | (+/−0.1) |
| (+/−SD) | Max index of | 46.6 | (+/−11) | 47.4 | (+/−13) |

phagocytosis

TABLE 7
In vitro assessment of EC50 and Emax for phagocytosis
activity of CEACAM5 × CD47 bispecific antibodies
K2AC100 and K2AC22 using SNU-C1 human cancer cell line
as target with 5 different donors (D) of macrophages.

| | CD47 × CEACAM5 | |
|---|---|---|
| | bispecific antibodies | |
| Donors (D) | K2AC22 | K2AC100 |

| D860 | EC50 | 0.02 | | 0.02 | |
|---|---|---|---|---|---|
| | (μg/mL) | | | | |
| | Max index of | 26 | | 22 | |
| | phagocytosis | | | | |
| D868 | EC50 | 0.6 | | 0.3 | |
| | (μg/mL) | | | | |
| | Max index of | 20 | | 21 | |
| | phagocytosis | | | | |
| D870 | EC50 | 0.07 | | 0.07 | |
| | (μg/mL) | | | | |
| | Max index of | 14 | | 14 | |
| | phagocytosis | | | | |
| D871 | EC50 | 0.96 | | 1.28 | |
| | (μg/mL) | | | | |
| | Max index of | 17 | | 17 | |
| | phagocytosis | | | | |
| D875 | EC50 | 0.18 | | 0.08 | |
| | (μg/mL) | | | | |
| | Max index of | 22 | | 16 | |
| | phagocytosis | | | | |
| Mean | EC50 | 0.37 | (+/−0.4) | 0.35 | (+/−0.53) |
| (+/−SD) | Max index of | 19.8 | (+/−4.6) | 18 | (+/−3.4) |

phagocytosis

TABLE 8
EC50 and Eₘₐₓ for phagocytosis activity of CEACAM5 ×
CD47 bispecific antibodies K2AC22 and K2AC100 on Ls174T
human cancer cell line with 5 different donors of macrophages.

| | CD47 × CEACAM5 | |
|---|---|---|
| | bispecific antibodies | |
| Donors (D) | K2AC22 | K2AC100 |

| D862 | EC50 | 0.25 | | 0.56 | |
|---|---|---|---|---|---|
| | (μg/mL) | | | | |
| | Max index of | 37 | | 46 | |
| | phagocytosis | | | | |
| D863 | EC50 | N/A | | N/A | |
| | (μg/mL) | | | | |
| | Max index of | 32 | | 40 | |
| | phagocytosis | | | | |
| D866 | EC50 | 1.57 | | 0.07 | |
| | (μg/mL) | | | | |
| | Max index of | 15 | | 14 | |
| | phagocytosis | | | | |
| D874 | EC50 | 0.33 | | 0.05 | |
| | (μg/mL) | | | | |
| | Max index of | 51 | | 48 | |
| | phagocytosis | | | | |
| D875 | EC50 | 0.25 | | 0.35 | |
| | (μg/mL) | | | | |
| | Max index of | 25 | | 35 | |
| | phagocytosis | | | | |
| Mean | EC50 | 0.6 | (+/−0.65) | 0.26 | (+/−0.24) |
| (+/−SD) | Max index of | 32 | (+/−13.5) | 36.6 | (+/−13.6) |

phagocytosis

TABLE 9
EC50 and Eₘₐₓ for phagocytosis activity of CEACAM5 ×
CD47 bispecific antibodies K2AC22 and K2AC100 on LoVo human
cancer cell line with 6 different donors of macrophages.

| | CD47 × CEACAM5 | |
|---|---|---|
| | bispecific antibodies | |
| Donors (D) | K2AC22 | K2AC100 |

| D862 | EC50 | 0.14 | | 0.054 | |
|---|---|---|---|---|---|
| | (μg/mL) | | | | |
| | Max index of | 30 | | 27 | |
| | phagocytosis | | | | |
| D863 | EC50 | 0.28 | | 0.37 | |
| | (μg/mL) | | | | |
| | Max index of | 19 | | 30 | |
| | phagocytosis | | | | |
| D866 | EC50 | 0.24 | | 0.08 | |
| | (μg/mL) | | | | |
| | Max index of | 29 | | 34 | |
| | phagocytosis | | | | |
| D872 | EC50 | 1.14 | | 2.34 | |
| | (μg/mL) | | | | |
| | Max index of | 10 | | 13 | |
| | phagocytosis | | | | |
| D873 | EC50 | N/A | | N/A | |
| | (μg/mL) | | | | |
| | Max index of | 11 | | 17 | |
| | phagocytosis | | | | |
| D874 | EC50 | 0.14 | | 0.052 | |
| | (μg/mL) | | | | |
| | Max index of | 30 | | 32 | |
| | phagocytosis | | | | |
| Mean | EC50 | 0.39 | (+/−0.42) | 0.58 | (+/−1) |
| (+/−SD) | Max index of | 21.5 | (+/−9.5) | 25.5 | (+/−8.5) |

phagocytosis

### Example 8: Production of Afucosylated Bispecific Antibodies of the Invention

Tables 10 and 11 show the results for the phagocytosis of two cell lines (MKN-45 and SNU-C1) by afucosylated versions of the bispecific antibodies of the invention (EC50 and Emax). The afucosylated versions of the bispecific antibodies of the invention have been produced and purified by the following methods:

### 1. Production

CHO pool transfected with the plasmid for the respective bispecific antibody of the invention (for vectors respectively plasmids see Example 2) is inoculated at a viable cell concentration of 0.3×10⁶ cells/mL in a Thomson erlen device with a working volume of 700 mL or 100 mL for the production of fucosylated and afucosylated antibodies, respectively. All the pools are operated in a 15 day duration fed-batch mode using CDACF medium CDCHO and an adapted feeding regime. To produce afucosylated antibodies, bolus of 200 μM fucose inhibitor (1,3,4-Tri-O-acetyl-2-deoxy-2-fluoro-L-fucose) are added at day 0, 5, 8 and 11 during the fed batch process, based on afucosylation strategy described by Rillahan et al. Nature Chem. Biol. 2012 July; 8(7):661-8 and based on EP2282773. Harvest of the bispecific antibodies of the invention pools supernatants containing fucosylated or afucosylated antibodies is performed after 15 days of Fed batch culture. Harvests of CHO pools supernatants are clarified using the Sartoclear Dynamics® Lab V Cell Harvesting Sartorius system (see supplier instructions).

### 2. Purification

Purification of fucosylated and afucosylated bispecific antibodies of the invention is a three-step affinity purification process. Before starting purification, antibody concentration in the supernatant of bispecific antibody pools is measured using OctetRED96 in order to use columns with appropriate volume of affinity matrix. Each clarified CHO pool supernatant containing fucosylated or afucosylated bispecific antibodies is loaded onto a MabSelect SuRe (MSS) column (GE Healthcare) without prior adjustment, to remove a major part of cell culture contaminants. The MSS eluate is then treated by low pH hold to inactivate viruses and neutralized at pH 6 with Tris 1M pH9. The MSS eluate is then loaded onto the LambdaFabSelect (LFS) column (GE Healthcare) to remove monospecific κ (mono κ). The LFS eluate is then pH adjusted at pH 6. The LFS is loaded onto the Capto L (CL) column (GE Healthcare) to remove monospecific λ (mono λ). The CL Eluate is pH adjusted before storage. The final material is then concentrated and diafiltered into the final formulation buffer, its concentration adjusted using the Nanodrop. Fucosylated and afucosylated bispecific antibodies are aliquoted and stored at −80° C. until use. Purified bispecific antibodies are analyzed for sizing by electrophoresis in denaturing and reducing conditions with the Agilent 2100 Bioanalyzer using the Protein 80 kit as described by the manufacturer (Agilent Technologies, Santa Clara, Calif, USA). Aggregation level is assessed by size exclusion chromatography (SEC-UPLC) using the ACQUITY UPLC H-Class Bio System (Waters). Charge variant analysis of purified bispecific antibodies is achieved by isoelectric focusing technique (JEF) using the Multiphor II Electrophoresis System (GE Healthcare). The relative distribution of N-linked complex biantennary glycoforms of fucosylated and afucosylated K2AC5 and K2AC22 antibodies is determined using the throughput microchip-CE method on the LabChip GXI Touch (Perkin Elmer). All antibodies are tested for endotoxin contamination using the Limulus Amebocyte Lysate test (LAL; Charles River Laboratories, Wilmington, Mass). The afuc bispecific antibodies of the invention showed afucosylation of >70%.

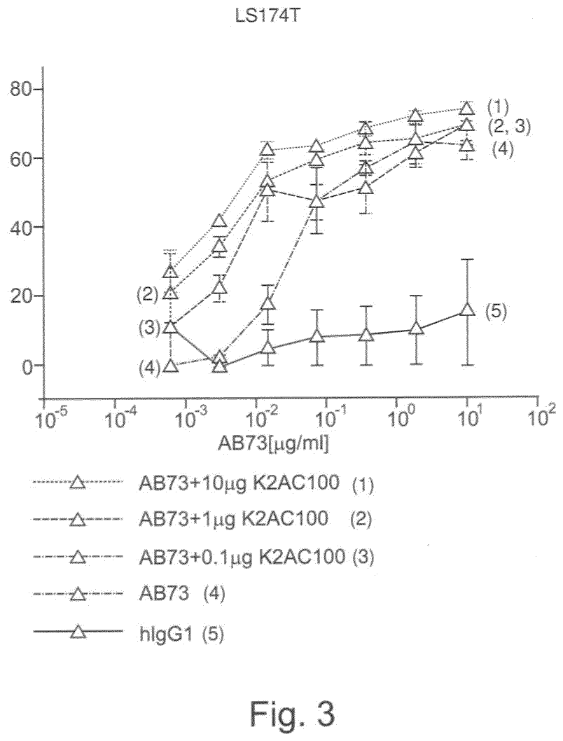

These afucosylated CEA×CD47 bispecific antibodies have been used to obtain the results shown in Tables 10 and 11 and in FIGS. 3A and 3B.

### 3. Other Methods to Produce Afucosylated Bispecific Antibodies of the Invention

### 3.1 by Using FUT 8 Negative Production Cell Line

Alternatively, and according to the knowledge of the inventors, afucosylated bispecific antibodies according to the invention can be produced also according to the method as follows:

Material and Methods are according to Naoko Yamane-Ohnuki et al., Biotech. Bioeng.; 87 (2004) 614-622.

Isolation of Chinese Hamster FUT8 cDNA

According to the knowledge of the inventors, total RNA is isolated from CHO/DG44 cells using the RNeasy® Mini Kit (Qiagen, Hilden, Germany) and reverse transcribed with oligo-dT using a Superscript first-strand synthesis system for reverse transcript-polymerase chain reaction (RT-PCR) (Invitrogen, Carlsbad, CA). A Chinese hamster FUT8 cDNA is amplified from single-stranded CHO/DG44 cell cDNAs by PCR using primers 5V-GTCTGAAGCATTATGTGTTGAAGC-3V (SEQ ID NO:44) and 5V-GTGAGTACATTCATTGTACTGTG-3V (SEQ ID NO:45), designed from the murine FUT8 cDNA (Hayashi, 2000; DNA Seq 11:91-96).

### Targeting Construct of FUT8 Locus

According to the knowledge of the inventors, the targeted disruption of the FUT8 gene in CHO/DG44 cells is carried out using two replacement vectors, pKOFUT8Neo and pKOFUT8Puro. The 9.0-kb fragment of the FUT8 gene including the first coding exon is isolated by screening the CHO-K1 cell E-genomic library (Stratagene, La Jolla, CA) with the Chinese hamster FUT8 cDNA as a probe to establish the targeting constructs. A 234-bp segment containing the translation initiation site is replaced with the neomycin-resistance gene (Neor) cassette or the puromycin-resistance gene (Puror) cassette from plasmid pKOSelectNeo or pKOSelectPuro (Lexicon, TX), respectively, flanked by loxP sites. The diphtheria toxin gene (DT) cassette from plasmid pKOSelectDT (Lexicon) is inserted at the 5V homologous region. The resulting targeting constructs, pKOFUT8Neo and pKOFUT8Puro, included the 1.5-kb 5V homologous sequence and the 5.3-kb 3V homologous sequence. Before transfection, the targeting constructs are linearized at a unique SalI site.

### Transfection and Screening for Homologous Recombinants

According to the knowledge of the inventors, subconfluent CHO/DG44 cells (1.6 106) are electroporated with 4 Ag of linearized pKOFUT8Neo at 350 V and 250 AF using a Bio-Rad GenePulser® II. After electroporation, transfectants are selected with 600 Ag/mL G418 (Nacalai Tesque, Kyoto, Japan). Genomic PCR is performed in 96-well plates by the modified microextraction method reported previously (Ramirez-Solis et al., 1992; Anal Biochem 201:331-335) using the following primers:

(SEQ ID NO: 40)
5V-TTGTGTGACTCTTAACTCTCAGAG-3V
and
(SEQ ID NO: 41)
5V-GAGGCCACTTGTGTAGCGCCAAGTG-3V.

Homologous recombinants are identified by the 1.7-kb fragment obtained using genomic PCR and confirmed by Southern blot analysis using the 221-bp fragment amplified with the following primers:

(SEQ ID NO: 42)
5V-GTGAGTCCATGGCTGTCACTG-3V
and
(SEQ ID NO: 43)
5V-CCTGACTTGGCTATTCTCAG-3V.

The hemizygous clone is subject to a second round of homologous recombination using linearized pKOFUT8Puro and drug selection with 15 Ag/mL puromycin (Sigma-Aldrich, St. Louis, MO) as described earlier. The identified homozygous disruptants are electroporated with the Cre-recombinase expression vector pBS185 (Invitrogen) to remove drug-resistance gene cassettes from both FUT8 alleles.

### Monoclonal Antibody Production by FUT8(−) Cells

According to the knowledge of the inventors, FUT8(−) cell lines are electroporated with an expression vector encoding a bispecific antibody according to the invention and selected in media lacking hypoxanthine and thymidine. The confluent transfectants are cultured in Ex-Cell® 301 Medium (JRH Biosciences, Lenexa, KS) for 1 week. The antibody is purified from culture supernatants using MabSelect™ (Amersham Biosciences, Piscataway, NJ). Further purification steps can be anion/cation exchange chromatography, size exclusion chromatography and especially purification using kappa respectively lambda selective resins as described above.

3.2. By Retrieval of Extracellular Fucose from Production Cell Medium Plus Enzymatic Intervention with the Intracellular Fucose Biosynthesis

Preferably, and according to the knowledge of the inventors, afucosylated bispecific antibodies of the invention can be produced also according to the method/technology as follows and described in U.S. Pat. No. 8,642,292. This technology is designed to configure the stable integration of a heterologous bacterial enzyme into an antibody producer cell line like a CHO cell line or others. By this, the de novo synthesis of fucose from D-mannose is blocked. If in addition production cells are cultivated in fucose free medium, as a result antibodies with a stable level of afucosylation are produced.

In eukaryotic cells fucose is generated through two routes,
a) from the extracellular space or lysosome through the salvage pathway and
b) by de novo synthesis of fucose from D-mannose in the de novo synthesis pathway of fucose.

The salvage pathway can be completely blocked by omission of fucose from the culture medium. The de novo biosynthesis pathway can be blocked by converting the intermediate GDP-4-keto-6-deoxy-D-mannose of this pathway to GDP-D-rhamnose instead of GDP-4-keto-6-deoxy-D-galactose. This is achieved by bringing the bacterial enzyme GDP-6-deoxy-D-lyxo-4-hexulose reductase (RMD) into the production cell line, respectively by stable integration of the gene encoding for RMD into the production cell line. Even rather low amounts of RMD expressed in the production cell line completely block the de novo synthesis pathway of the production cell.

This technology will be used to construct production cell lines, e.g. CHO based cell lines, designed for the production of afucosylated antibodies of the invention, as well as to existing production cell lines which already produce antibodies of the invention and are engineered to produce the antibodies with fucose content reduced by 80% to 100%.

All results shown in FIGS. 3A and 3B and Tables 10 and 11 are obtained with 2 CEACAM5-expressing cancer cell lines (MKN-45 and SNU-C1) with an effector cell to target/tumor cell ratio of 1:3. These results are obtained in experiments using macrophages obtained from three different human donors. The data obtained from such experiments are shown in Table 10 (for MKN-45 cells) and Table 11 (for SNU-C1 cells).

TABLE 10
In vitro assessment of EC50 (μg/mL) and Eₘₐₓ (max index
of phagocytosis) from phagocytosis activity of afucosylated
CEACAM5 × CD47 bispecific antibodies K2AC100 afuco,
and K2AC22 afuco (comparison)) using MKN45 human cancer
cell line as target with 2 different donors (D) of macrophages.

| | CD47 × CEACAM5 | |
|---|---|---|
| | bispecific antibodies | |
| | K2AC22 | K2AC100 |
| Donors (D) | afuco | afuco |

| D830 | EC50 | 0.11 | | 0.14 | |
|---|---|---|---|---|---|
| | (μg/mL) | | | | |
| | Max index of | 62 | | 84 | |
| | phagocytosis | | | | |
| D831 | EC50 | 0.26 | | 0.08 | |
| | (μg/mL) | | | | |
| | Max index of | 34 | | 37 | |
| | phagocytosis | | | | |
| Mean | EC50 | 0.2 | (+/−0.11) | 0.1 | (+/−0.04) |
| (+/−SD) | Max index of | 48 | (+/−19.8) | 60.5 | (+/−33) |

phagocytosis

TABLE 11
In vitro assessment of EC50 (μg/mL) and Eₘₐₓ from
phagocytosis activity of afucosylated CEACAM5 ×
CD47 bispecific antibodies K2AC100 afuco and K2AC22
afuco (comparison)) using SNU-C1 human cancer cell
line as target with 2 different donors (D) of macrophages.

| | | CD47 × CEACAM5 | | | |
|---|---|---|---|---|---|
| | | bispecific antibodies | | | |
| | | K2AC22 | | K2AC100 | |
| Donors (D) | | afuco | | afuco | |
| D831 | EC50 | 0.2 | | 0.08 | |
| | (μg/mL) | | | | |
| | Max index of | 31 | | 32 | |
| | phagocytosis | | | | |
| D833 | EC50 | 0.09 | | 0.027 | |
| | (μg/mL) | | | | |
| | Max index of | 32 | | 38 | |
| | phagocytosis | | | | |
| Mean | EC50 | 0.15 | (+/−0.08) | 0.05 | (+/−0.04) |
| (+/−SD) | Max index of | 31.5 | (+/−0.7) | 32 | (+/−1.4) |

phagocytosis

### Example 9

Blocking the Interaction of SIRPα with CD47 on Tumor Cells.

Experimental set-up for the measurement of the SIRPα inhibition potency (IC50) of the bispecific antibodies of this invention:

The cell-based assay monitoring the interaction of soluble SIRPα with human CD47 expressed at the surface of MKN-45 cells as described below is used for the detection of the blocking activity. Concentration-response experiments with bispecific antibodies according to the invention allow determination of inhibition curves (see FIG. 4) and of IC50 values (see Table 12).

MKN-45 cancer cells, expressing both CD47 and CEACAM5, are stained with CFSE violet for detection of e.g. cellular division by the imaging system (CX5). Briefly, 3′000 stained MKN-45 cells per well are seeded in a 384 optical well plate (Costar) and incubated for 50 minutes with increased concentrations of bispecific antibodies of the invention (1.9 pM to 333 nM, in quadruplicates). Then, a fix concentration of SIRPα-mouseFc premixed with anti-mouse IgG-Fc AF647 coupled antibody (Jackson Immunoresearch diluted 1:2000) is added at 50 ng/mL final. After an incubation of 3H30 plates are acquired with the imaging system (CX5, Thermo Fisher) and fluorescence signals emitted by the detected bound SIRPα is recorded by the software dedicated to the imaging system. Fluorescence signals (mean fluorescence intensity MFI) are plotted according to the dose range tested and IC50 are calculated by the software (Prism, GraphPad).

Results are shown in Table 12:

TABLE 12
IC50 (nM) measured with the CD47/SIRPα blocking
assay for CEACAM5 × CD47 bispecific antibodies
K2AC100 and K2AC22 (using MKN-45 as hCD47-expressing cells).

| Antibody | SIRPα inhibition |
|---|---|
| name | potency (nM)^{#} |
| K2AC22 | 1.2 |
| K2AC100 | 0.09 |

### Example 10: Organoid Procedure to a. Obtain CEACAM5 Expression in Cancer Cells from Fresh Samples from Cancer Patients (Qifikit Data) and b. To Obtain Phagocytosis Data

Organoids derived from primary samples of patients were prepared as single cell suspension by standard methods (enzymatic digestion and/or mechanical dissociation). 10 μL of anti-human CEACAM5 primary antibody ((#sc-23928; mIgG1 (Santa Cruz); final concentration 20 g/mL) were added to the cells and incubated 30 min at 4° C. Cells were washed, and centrifuged. 100 μL of beads (Setup or Calibration from QIFIKIT®) were washed along with the cells and treated identically. 100 μL of secondary antibody from the kit (1/50 in PBS BSA 2%) were added to each well and incubated for 30 to 45 min at 4° C. Cells were centrifuged to discard the supernatant and washed twice. After the last centrifugation, cells were resuspended and assessed using a flow cytometer. Analysis was done using specific software and Geometric means exported to an Excel file. A linear regression was performed using MFI values from the calibration beads. Antibody Binding Capacity (ABC) of cells were extrapolated from this regression line. Specific Antibody Binding Capacity (sABC) was obtained by subtracting ABC from isotype control to the one of the specific staining.

The average expression of CEACAM5 of these primary organoids has been found to be 28,000 CEACAM5 targets per cell, i.e. a factor of approximately 4-fold lower than average expression on the cell lines in Table 5.

The organoids derived from primary samples of cancer patients can also be used to study concentration dependent phagocytosis/phagocytosis index if bispecific antibodies of the invention and macrophages from human donors are added (see Example 7). By using the same methods, according to the knowledge of the inventors, also combinations of the bispecific antibodies of the invention with CEA×CD3 bispecific antibodies can be studied if also T-cells from human donors are added.

### Example 11: Mixed Killing Assay by Combination of CEA×CD3 and CEA×CD47

Human peripheral blood mononuclear cells (PBMCs) were isolated from buffy coats (healthy donors). An aliquot of these PBMCs was kept frozen in medium containing 90% FCS and 10% of cryoprotectant agent to be used as a source of T cells for the mixed killing assay. A further aliquot of the PBMCs was used to prepare macrophages derived from monocytes.

Briefly, 107 PBMCs are seeded in a 175 cm² in culture flask in medium containing 10% of decomplemented FCS and supplemented with 10% of AB+ human serum and 20 ng/mL of human M-CSF. After 24 h of incubation at 37° C., 5% of CO₂ the medium is removed to eliminate floating cells and replace by the culture medium only supplemented with 20 ng/mL-1 of human M-CSF. After 2 additional days of incubation the half part of the medium is replaced by fresh culture medium supplemented with human M-CSF.

After 6 days of differentiation, the derived macrophages were plated in clear flat bottom 96 well-plates and incubated at 37° C. Two days after the plating, the frozen PBMCs from the corresponding macrophage donor were thawed and added to the macrophage plates.

Target tumor cells (e.g. MKN45 or LS174T or other CEA positive tumor cells) were stained with Cell-Trace Violet and opsonized with the combination of the invention. Opsonized target cells were added to the plate containing macrophages and the corresponding autologous PBMCs. The plates were incubated for 48 hours (or 72H) at 37° C.

After the incubation period, floating cells in supernatant were collected and the adherent cells were detached with trypsin and recovered with centrifugation. Floating and attached cells recovered from the same well were pooled and stained with a labelled anti-CD14 antibody to identify the macrophages during the cytometry analysis. At the end of the staining, a viability marker was added (sytox green) to eliminate the dead cells from the analysis.

The cells were analyzed with a Flow Cytometer (Cytoflex/Beckman). Acquired events were normalized to analyze the same number of events for each assay condition. The number of live target cells were calculated as, live target cells=total Cell-trace Violet positive live cell-CD14/Cell-Trace Violet double positive live cells. The percentage of killing for each condition was calculated as (1-(live target cells in the treated well/live target cells in negative control well))×100%.

The combination of the invention was studied in two different set-ups:
1. Concentration response curves for AB73 were established with no K2AC100 added as well as combined with defined concentration(s) of K2AC100 (see FIG. 1)
2. Concentration response curves for K2AC100 were established with no AB73 added as well as combined with defined concentration(s) of AB73L3-1/N (see FIG. 5)

Results are shown in the FIGS. 1, 2, 3, 4, 5, and 6.

FIG. 1 shows the results of an experiment run in set-up number 1. AB73 achieved at highest concentration (saturation of the concentration response curve)) approx. 30% killing, combination with 1 or 10 μg/ml K2AC100 increased the effect to 80% killing. FIG. 2 shows from the same study the effects achieved with 1 as well as 10 μg/ml K2AC100 in monotherapy, the % of killing was clearly below the % killing achieved with the combinations shown in FIG. 2.

FIG. 3 shows results again at set-up number 1, but with a different donor and different tumor cells (LS-174T instead of MKN-45). Monotherapy with AB73 achieved already approx. 70% of killing at maximum (saturation of the concentration response curve), combination with K2AC100 increased to approx. 80% of killing. These approx. 80% of killing are also achieved at lower concentrations of AB73 if K2AC100 is added—see FIG. 4. K2AC100 in monotherapy did not, in the concentrations tested, i.e. 1 and 10 μg/ml, achieve more than 40% of killing.

FIGS. 5 and 6 show results from a study in set-up number 2. The PBMC and macrophages of the donor used in this experiment caused already close to 100% of killing in K2AC100 monotherapy (FIG. 5). Combination brought about the same maximal killing. But again, combinations with lower concentrations of the bispecific antibodies achieved higher percentage of killing than achieved with the two bispecific antibodies in monotherapy at the same concentrations.

### Example 12: Anti-Tumor Activity: Tissue Slice Cultures

Anti-tumor activity of a combination treatment with K2AC100 and AB73L3-1/N, in tumor tissue slice cultures from patients diagnosed with CEA-expressing tumors was performed according to the method described by Sönnichsen R. et al., Clinical Colorectal Cancer, Vol. 17, No. 2, e189-99, 2018).

### 1. Tissue Slice Culture and Treatment

Fresh tumor tissue samples are cut and handled as described by Sönnichsen R. et al. In brief, immediately after surgical resection and first macroscopic pathologic assessment, tumor samples are cut into slices of 350 μm using a tissue chopper. Tissue slice diameter is then standardized by using a 3-mm coring tool. Three tissue slices are randomly pooled, placed on membrane inserts, and cultivated in 6-well plates. Slices are incubated under standardized conditions of 37° C. and 5% CO2. After pre-cultivation in standard cell culture medium, slice triplets are exposed to the bispecific antibodies alone or in combination and in combination with PD-L1 inhibitors, for up to 120 hours. After compound exposure, tumor slices are fixed overnight using 4% paraformaldehyde.

### 2. Staining

Paraformaldehyde fixed slices are embedded in paraffin and processed to 5-μm sections. Hematoxylin & eosin (HE) staining is performed to assess histopathologic aspects and tumor cell proportion. Overall cell count, tumor cell count, and proliferation are analyzed by immunofluorescent staining. In brief, paraffin sections are deparaffinized. After antigen retrieval, sections are washed with 0.3% PBS/TritonX and blocked with 5% normal goat serum for 30 minutes. Primary antibodies against cytokeratins (AE1♭3), Ki67, and cleaved-PARP, respectively, are diluted in 0.5% bovine serum albumin and incubated at 4° C. overnight. Sections are rinsed with 0.3% phosphate buffered saline/TritonX and labeled with secondary antibodies. Nuclei are stained with Hoechst 33342. Additional staining (e.g. for CEA expression) may be included.

### 3. Data Analysis

Five pictures (20×) per tissue slice are taken from fluorescent-stained sections using a fluorescent microscope. The positive pixel count is determined for Hoechst 33342, cytokeratin, Ki67, and cleaved-PARP stains with stain-specific segmentation algorithms. Proliferating/apoptotic tumor area is calculated by analyzing pixels of Ki67/cleaved PARP positive nuclei surrounded by cytokeratin-positive pixels. For every picture, the total cell count (Hoechst-positive), tumor cell count (Hoechst- and cytokeratin-positive), and proliferating tumor cell count (Hoechst-, cytokeratin, and Ki67-positive/cleaved-PARP) is calculated. Tumor cell count is normalized to total cell count and proliferating tumor cell count is normalized to tumor cell count to consider different tumor cell fractions per picture. Mean slice values are then calculated from single image values. Mean values for conditions are calculated using mean slice values. Preliminary data support a beneficial impact of the combination of K2AC100 and AB73L3-1/N for patients with CEA-expressing solid tumors.

### Example 13: In Vivo Anti-Tumor Activity of CEA×CD47 in Combination with CEA×CD3 Bispecific Antibodies in Human PBMCs and Macrophages Co-Engraftment Mouse Tumor Model

According to the knowledge of the inventors the antitumor activity of the combination of the invention can be evaluated in the following mouse model:

Human peripheral blood mononuclear cells (PBMCs) were isolated from buffy coats (healthy donors). A part of these PBMCs were kept frozen in medium containing 90% FCS and 10% of cryoprotectant agent and will be used the day of the engraftment. The main part of the PBMCs were used to prepare macrophages derived from monocytes. Briefly, 10⁷ PBMCs are seeded in a T175 cm² culture flasks in culture medium containing 10% of decomplemented FCS and supplemented with 10% of AB+ human serum and 20 ng/mL of human M-CSF. After 24 h of incubation at 37° C., 5% of CO₂ the medium is removed to eliminate floating cells and replace by the culture medium only supplemented with 20 ng/mL-1 of human M-CSF. After 2 additional days of incubation the half part of the medium is replaced by fresh culture medium supplemented with human M-CSF. After 6 days of differentiation, the macrophages were detached and resuspended in PBS.

The frozen PBMCs were thawed and resuspended in sterile PBS.

In the meantime, the CEA expressing target cells (LS174T or HPAF-II or other CEA positive tumor cells) in exponential growing phase were detached and the cell suspension adjusted in PBS. The cell suspensions were counted by using an automated trypan blue based cell counter (ViCell XR #Beckman).

Finally, the three different cell suspensions were mixed to be used for the engraftment. The final mixed cell suspension contained in 100 μL 10⁶ CEA expressing target cells, 10⁶ PBMCs and the same number or alternatively a higher or lower number of human macrophages. NSG mice (or alternatively NOG mice) are engrafted subcutaneously with 100 μL of the cell suspension containing the LS174T or other tumor cells, human PBMCs and the human macrophages.

Day +1 or later post engraftment the mice in dedicated groups were injected first time with either CEA×CD3 alone or in combination with the CEA×CD47 molecule. During the following weeks, the mice are injected with the combination of the invention, e.g. twice or once a week i.v. In parallel, control groups are injected only with each single molecule. Finally, a group is injected with the vehicle to get tumor growth reference curve. Mice are monitored for tumor growth at least two times a week and tumors are measured by digital caliper until the endpoint of the experiment (tumor volume=1500 mm³ or occurrence of GvHD symptoms). Tumor volume is calculated using the formula (length×width)×0.5. Statistical analysis is performed using one-way ANOVA comparison analysis at study.

### Example 14: In Vivo Anti-Tumor Activity in a Transgenic Mouse Model

According to the knowledge of the inventors, anti-tumor activity of the combination according to the invention can be evaluated as single agent as well as in combination treatment, respectively, in transgenic mice.

### 1. Cell Line Generation and Growth Testing

A hCEACAM5(Tg)hCD47(Tg)mCD47(ko) cell line, e.g. based on the murine colon cancer cell lines CT26 or MC38, will be generated. Knock-out (KO) of the endogenous mouse CD47 gene is performed by using CRISPR/Cas9 with subsequent isolation of KO clones by cell sorting. Transfection of the KO clones with a cassette driving the expression of both hCD47 and hCEACAM5 using an internal ribosome entry site (IRES) is performed followed by isolation of engineered clones based on e.g. overall expression levels and ratio. Three validated clones will be selected to subsequently test their engraftment/tumorigenicity in vivo for selection of the final clone.

### 2. In Vivo Anti-Tumor Activity

Mice strains of BALB/cJGpt background expressing human CD3e (T001550 heterozygous BALB/c-hCD3ET/Wt mice) and human CD47/human SIRPα (T037264 homozygous BALB/c-hCD47/hSIRPα mice) are available at GemPharmatech. Alternatively, mice strains of C57BL/6/Bcgen background expressing human CD3e (homozygous B-hCD3E mice) and human CD47/human SIRPα (homozygous B-hSIRPα/hCD47 mice) are available at Biocytogen. In each case, the two mouse strains will be crossed to obtain triple humanized hCD3e/hSIRPα/hCD47 mice, and the offspring used for subsequent experiments to test a bispecific antibody according to the invention either as single agent or in combination treatment.

Triple humanized hCD3e/hSIRPα/hCD47 mice are inoculated with either CT26-hCEACAM5(Tg)hCD47(Tg)mCD47(ko) cell line (BALB/c background) or MC38-hCEACAM5(Tg)hCD47(Tg)mCD47(ko) cell line (C57BL/6 background) at day 0. Once medium tumor size in the cohort reaches e.g. 200 mm³, treatment with a bispecific antibody according to the invention as single agent as well as in combination is initiated as i.v. bolus at an interval of e.g. 2 treatments/week until one mouse shows a tumor volume of e.g. over 3000 mm³ or any one or more of the pre-specified animal protection and care endpoints occur. Tumor volume and body weight are measured three times per week. Tumor volume is given in mm³ using the following formula: TV=0.5 a×b², where a and b are the long and short diameters of the tumor, respectively.

1. A first bispecific antibody, comprising a first binding part specifically binding to human CEACAM5 and a second binding part specifically binding to human CD47, characterized in that
a) the first binding part comprises a heavy chain variable region comprising a CDRH1 of SEQ ID NO:23, a CDRH2 of SEQ ID NO:24 and a CDRH3 of SEQ ID NO:25, and a light chain variable region comprising a CDRL1 of SEQ ID NO:35, CDRL2 of SEQ ID NO:36, and CDRL3 of SEQ ID NO:37,
b) the second binding part comprises a heavy chain variable region comprising a CDRH1 of SEQ ID NO:23, a CDRH2 of SEQ ID NO:24, and a CDRH3 of SEQ ID NO:25,
and as light chain variable region a light chain variable region comprising a CDRL1 of SEQ ID NO:29, a CDRL2 of SEQ ID NO:30, and a CDRL3 of SEQ ID NO:31,
for use in the treatment of cancer in combination with
a second bispecific antibody comprising a first binding part specifically binding to human CEACAM5 and a second binding part specifically binding to human CD3ε, characterized in that:
a) the first binding part comprises a heavy chain variable region comprising a CDRH1 of SEQ ID NO:2, a CDRH2 of SEQ ID NO:3 and a CDRH3 of SEQ ID NO:4, and a light chain variable region comprising a CDRL1 of SEQ ID NO: 18, CDRL2 of SEQ ID NO:19, and CDRL3 of SEQ ID NO:20, and
b) the second binding part comprises a heavy chain variable region comprising a CDRH1 of SEQ ID NO:2, a CDRH2 of SEQ ID NO:3 and a CDRH3 of SEQ ID NO:4, and a light chain variable region comprising a CDRL1 of SEQ ID NO:6, a CDRL2 of SEQ ID NO:7, and a CDRL3 of SEQ ID NO:8.

2. A first bispecific antibody comprising a first binding part specifically binding to human CEACAM5 and a second binding part specifically binding to human CD47 characterized in that:
a) the first binding part comprises a heavy chain variable region of SEQ ID NO:26 and a light chain variable region of SEQ ID NO:38, and
b) the second binding part comprises a heavy chain variable region of SEQ ID NO:26, and a light chain variable region of SEQ ID NO:32,
for use in the treatment of cancer in combination with a second bispecific antibody comprising a first binding part specifically binding to human CEACAM5 and a second binding part specifically binding to human CD3ε characterized in that:
a) the first binding part comprises a heavy chain variable region of SEQ ID NO:1 and a light chain variable region of SEQ ID NO:21, and
b) the second binding part comprises a heavy chain variable region of SEQ ID NO: 1, and a light chain variable region of SEQ ID NO:5.

3. A first bispecific antibody comprising a first binding part specifically binding to human CEACAM5 and a second binding part specifically binding to human CD47 characterized in that:
a) the first binding part comprises a heavy chain region of SEQ ID NO:27 and a light chain of SEQ ID NO: 39, and
b) the second binding part comprises a heavy chain region of SEQ ID NO:27, and a light chain of SEQ ID NO:33, for use in the treatment of cancer in combination with
a second bispecific antibody comprising a first binding part specifically binding to human CEACAM5 and a second binding part specifically binding to human CD3ε characterized in that:
a) the first binding part comprises a heavy chain region of SEQ ID NO:15 and a light chain of SEQ ID NO:22, and
b) the second binding part comprises a heavy chain region of SEQ ID NO:15, and a light chain of SEQ ID NO:9.

4. The bispecific antibody for use according to anyone of claims 1 to 3, characterized in that the cancer is a solid cancer.

5. The bispecific antibody for use according to claim 4, characterized in that the cancer is a colorectal cancer, NSCLC (non-small cell lung cancer), gastric cancer, pancreatic cancer, breast cancer, or another CEACAM5 expressing cancer.

6. The first bispecific antibody for use according to anyone of claims 1 to 3, characterized by a ratio of the KD values for the binding to recombinant CEACAM3 and recombinant CEACAM5 of a factor of 100 or more.

7. The first bispecific antibody for use according to anyone of claims 1 to 3, characterized by a ratio of the KD values for the binding to recombinant CEACAM3 and recombinant CEACAM5 of a factor of between 100 and 200.

8. The bispecific antibody for the use according to anyone of claims 1 to 7, characterized in that the first bispecific antibody and the second bispecific antibody are administered to said subject simultaneously in 2 to 15 day intervals.

9. A combination of a first bispecific antibody comprising a first binding part specifically binding to human CEACAM5 and a second binding part specifically binding to human CD47, characterized in that:
a) the first binding part comprises a heavy chain variable region comprising a CDRH1 of SEQ ID NO:23, a CDRH2 of SEQ ID NO:24 and a CDRH3 of SEQ ID NO:25, and a light chain variable region comprising a CDRL1 of SEQ ID NO:35, CDRL2 of SEQ ID NO:36, and CDRL3 of SEQ ID NO:37,
b) the second binding part comprises a heavy chain variable region comprising a CDRH1 of SEQ ID NO:23, a CDRH2 of SEQ ID NO:24, and a CDRH3 of SEQ ID NO:25,
and as light chain variable region a light chain variable region comprising a CDRL1 of SEQ ID NO:29, a CDRL2 of SEQ ID NO:30, and a CDRL3 of SEQ ID NO:31, and
a second bispecific antibody comprising a first binding part specifically binding to human CEACAM5 and a second binding part specifically binding to human CD3ε characterized in that:
a) the first binding part comprises a heavy chain variable region comprising a CDRH1 of SEQ ID NO:2, a CDRH2 of SEQ ID NO:3 and a CDRH3 of SEQ ID NO:4, and a light chain variable region comprising
a CDRL1 of SEQ ID NO:18, CDRL2 of SEQ ID NO:19, and CDRL3 of SEQ ID NO:20, and b) the second binding part comprises a heavy chain variable region comprising a CDRH1 of SEQ ID NO:2,
a CDRH2 of SEQ ID NO:3 and a CDRH3 of SEQ ID NO:4, and a light chain variable region comprising a CDRL1 of SEQ ID NO:6, a CDRL2 of SEQ ID NO:7, and a CDRL3 of SEQ ID NO:8.

10. A combination of a first bispecific antibody comprising a first binding part specifically binding to human CEACAM5 and a second binding part specifically binding to human CD47 characterized in that:
a) the first binding part comprises a heavy chain variable region of SEQ ID NO:26 and a light chain variable region of SEQ ID NO:38, and
b) the second binding part comprises a heavy chain variable region of SEQ ID NO:26, and a light chain variable region of SEQ ID NO:32, and
a second bispecific antibody comprising a first binding part specifically binding to human CEACAM5 and a second binding part specifically binding to human CD3ε characterized in that:
a) the first binding part comprises a heavy chain variable region of SEQ ID NO:1 and a light chain variable region of SEQ ID NO:21, and
b) the second binding part comprises a heavy chain variable region of SEQ ID NO: 1, and a light chain variable region of SEQ ID NO:5.

11. A combination of a first bispecific antibody comprising a first binding part specifically binding to human CEACAM5 and a second binding part specifically binding to human CD47 characterized in that:
a) the first binding part comprises a heavy chain region of SEQ ID NO:27 and a light chain of SEQ ID NO: 39, and b) the second binding part comprises a heavy chain region of SEQ ID NO:27, and a light chain of SEQ ID NO:33, and
a second bispecific antibody comprising a first binding part specifically binding to human CEACAM5 and a second binding part specifically binding to human CD38 characterized in that:
a) the first binding part comprises a heavy chain region of SEQ ID NO: 15 and a light chain of SEQ ID NO:22, and b) the second binding part comprises a heavy chain region of SEQ ID NO: 15, and a light chain of SEQ ID NO:9.

12. A pharmaceutical composition comprising the combination of anyone of claims 9 to 11 and a pharmaceutically acceptable excipient or carrier.

13. The pharmaceutical composition of claim 12, for use as a medicament.

14. The pharmaceutical composition of claim 12 or 13 for use as a medicament in the treatment of solid cancer.

15. The pharmaceutical composition of anyone of claims 12 to 14, for use as a medicament in the treatment of colorectal cancer, NSCLC (non-small cell lung cancer), gastric cancer, pancreatic cancer, or breast cancer.

16. A method of treating a patient having a cancer that expresses CEACAM5, the method comprising administering to the subject a therapeutically effective amount of the combination of anyone of claims 9 to 11 or the pharmaceutical composition of any one of claims 12 to 15.

17. The method of claim 16, wherein the antibodies are administered simultaneously.

18. The method of claim 16 or 17, wherein the patient is administered one or more doses of 0.01 to 10 mg/kg of the combination or composition of any one of claims 9 to 15.

19. The method of claim 16 or 17, wherein the patient is administered one or more doses of 0.01 to 10 mg/kg of the second bispecific antibody and one or more doses of 1 to 20 mg/kg of the first bispecific antibody.

20. A method of increasing survival time in a patient having a cancer that expresses CEACAM5, said method comprising administering to said patient a therapeutically effective amount of the combination or composition of any one of claims 7 to 11.

21. The combination of anyone of claims 9 to 11 for use in the manufacture of a medicament for treating a patient having a cancer that expresses CEACAM5.


SEQUENCE LISTING (45 sequences)

SEQ ID NO: 1 - protein, 125 aa, synthetic construct
  REGION 1..125: note = Common VH
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSS

SEQ ID NO: 2 - protein, 5 aa, synthetic construct
  REGION 1..5: note = Common CDRH1
TYAMN

SEQ ID NO: 3 - protein, 19 aa, synthetic construct
  REGION 1..19: note = Common CDRH2
RIRSKYNNYATYYADSVKD

SEQ ID NO: 4 - protein, 14 aa, synthetic construct
  REGION 1..14: note = Common CDRH3
HGNFGNSYVSWFAY

SEQ ID NO: 5 - protein, 109 aa, synthetic construct
  REGION 1..109: note = huCD3 VL 1A4
QTVVTQEPSLTVSPGGTVTLTCRSSTGAVTTSNYANWFQQKPGQAPRGLIGGTNKRAPGTPARFSGSLLGGKAALTLSGAQPEDEAEYYCALWYKQRWVFGGGTKLTVL

SEQ ID NO: 6 - protein, 14 aa, synthetic construct
  REGION 1..14: note = huCD3 1A4 CDRL1
RSSTGAVTTSNYAN

SEQ ID NO: 7 - protein, 7 aa, synthetic construct
  REGION 1..7: note = huCD3 1A4 CDRL2
GTNKRAP

SEQ ID NO: 8 - protein, 9 aa, synthetic construct
  REGION 1..9: note = huCD3 1A4 CDRL3
ALWYKQRWV

SEQ ID NO: 9 - protein, 215 aa, synthetic construct
  REGION 1..215: note = huCD 1A4 LC
QTVVTQEPSLTVSPGGTVTLTCRSSTGAVTTSNYANWFQQKPGQAPRGLIGGTNKRAPGTPARFSGSLLGGKAALTLSGAQPEDEAEYYCALWYKQRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 329 aa, synthetic construct
  REGION 1..329: note = Common constant heavy chain (WT IgG1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 117 aa, synthetic construct
  REGION 1..117: note = kappa light chain constant region (CK)
FGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 116 aa, synthetic construct
  REGION 1..116: note = lambda light chain constant region (CL)
FGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 454 aa, synthetic construct
  REGION 1..454: note = Common heavy chain (wild-type)
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 454 aa, synthetic construct
  REGION 1..454: note = Common heavy chain (LALA mutation)
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 454 aa, synthetic construct
  REGION 1..454: note = Common heavy chain (LALA+P329A mutation)
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 118 aa, synthetic construct
  REGION 1..118: note = hybrid-kappa light chain constant region (H-CK 5)
FGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 217 aa, synthetic construct
  REGION 1..217: note = huCD3 1A4 LC-hybrid kappa
QTVVTQEPSLTVSPGGTVTLTCRSSTGAVTTSNYANWFQQKPGQAPRGLIGGTNKRAPGTPARFSGSLLGGKAALTLSGAQPEDEAEYYCALWYKQRWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 11 aa, synthetic construct
  REGION 1..11: note = CEA AB73 CDRL1
RASQSVNSNLN

SEQ ID NO: 19 - protein, 7 aa, synthetic construct
  REGION 1..7: note = CEA AB73 CDRL2
HSNNRPH

SEQ ID NO: 20 - protein, 11 aa, synthetic construct
  REGION 1..11: note = CEA AB73 CDRL3
QQFDYFREYNT

SEQ ID NO: 21 - protein, 109 aa, synthetic construct
  REGION 1..109: note = CEA AB73 VL light chain variable region
EIVLTQSPATLSLSPGERATLSCRASQSVNSNLNWYQQKPGQAPRLLIYHSNNRPHGIPARFSGSGSGTDFTLTISSLEPEDFAVYYCQQFDYFREYNTFGQGTKVEIK

SEQ ID NO: 22 - protein, 216 aa, synthetic construct
  REGION 1..216: note = CEA AB73 LC light chain
EIVLTQSPATLSLSPGERATLSCRASQSVNSNLNWYQQKPGQAPRLLIYHSNNRPHGIPARFSGSGSGTDFTLTISSLEPEDFAVYYCQQFDYFREYNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, synthetic construct
  REGION 1..5: note = Common heavy chain CDRH1
SYAMS

SEQ ID NO: 24 - protein, 17 aa, synthetic construct
  REGION 1..17: note = Common heavy chain CDRH2
AISGSGGSTYYADSVKG

SEQ ID NO: 25 - protein, 7 aa, synthetic construct
  REGION 1..7: note = Common heavy chain CDRH3
SYGAFDY

SEQ ID NO: 26 - protein, 116 aa, synthetic construct
  REGION 1..116: note = Common heavy chain variable region VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 27 - protein, 229 aa, synthetic construct
  REGION 1..229: note = Common heavy chain (VH-CH1)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 28 - protein, 445 aa, synthetic construct
  REGION 1..445: note = common heavy chain (VH-CH1-CH2-CH3)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 11 aa, synthetic construct
  REGION 1..11: note = CD47 binding part CDRL1
RASQSISSYLN

SEQ ID NO: 30 - protein, 7 aa, synthetic construct
  REGION 1..7: note = CD47 binding part CDRL2
AASSLQS

SEQ ID NO: 31 - protein, 10 aa, synthetic construct
  REGION 1..10: note = CD47 binding part CDRL3
QQMHPRAPKT

SEQ ID NO: 32 - protein, 108 aa, synthetic construct
  REGION 1..108: note = CD47 binding part light chain
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIK

SEQ ID NO: 33 - protein, 215 aa, synthetic construct
  REGION 1..215: note = CD47 binding part light chain (VKCK; K2)
DIQMTQSPSSLSASVGDRVTITCRASQSISSYLNWYQQKPGKAPKLLIYAASSLQSGVPSRFSGSGSGTDFTLTISSLQPEDFATYYCQQMHPRAPKTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 648 bp, synthetic construct
  misc_feature 1..648: note = CD47 binding part light chain (VKCK; nucleic acid); (K2)
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagatgcacccgcgcgccccgaagaccttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 35 - protein, 13 aa, synthetic construct
  REGION 1..13: note = CEACAM5 binding part AC100 CDRL1
SGSSSNIGYGLVS

SEQ ID NO: 36 - protein, 7 aa, synthetic construct
  REGION 1..7: note = CEACAM5 binding part AC100 CDRL2
NGNIRPS

SEQ ID NO: 37 - protein, 10 aa, synthetic construct
  REGION 1..10: note = CEACAM5 binding part AC100 CDRL3
GTWDFSYRVD

SEQ ID NO: 38 - protein, 109 aa, synthetic construct
  REGION 1..109: note = CEACAM5 binding part light chain variable region AC100 VL
QSVLTQPPSVSAAPGQKVTISCSGSSSNIGYGLVSWYQQLPGTAPKLLIYNGNIRPSGIPDRFSGSKSGTSATLGITGLQTGDEADYYCGTWDFSYRVDFGGGTKLTVL

SEQ ID NO: 39 - protein, 215 aa, synthetic construct
  REGION 1..215: note = CEACAM5 binding part AC100 light chain VLCL
QSVLTQPPSVSAAPGQKVTISCSGSSSNIGYGLVSWYQQLPGTAPKLLIYNGNIRPSGIPDRFSGSKSGTSATLGITGLQTGDEADYYCGTWDFSYRVDFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - DNA, 24 bp, synthetic construct
  misc_feature 1..24: note = Primer
  source 1..24: mol_type = other DNA
ttgtgtgactcttaactctcagag

SEQ ID NO: 41 - DNA, 25 bp, synthetic construct
  misc_feature 1..25: note = Primer
  source 1..25: mol_type = other DNA
gaggccacttgtgtagcgccaagtg

SEQ ID NO: 42 - DNA, 21 bp, synthetic construct
  misc_feature 1..21: note = Primer
  source 1..21: mol_type = other DNA
gtgagtccatggctgtcactg

SEQ ID NO: 43 - DNA, 20 bp, synthetic construct
  misc_feature 1..20: note = Primer
  source 1..20: mol_type = other DNA
cctgacttggctattctcag

SEQ ID NO: 44 - DNA, 24 bp, synthetic construct
  misc_feature 1..24: note = Primer
  source 1..24: mol_type = other DNA
gtctgaagcattatgtgttgaagc

SEQ ID NO: 45 - DNA, 23 bp, synthetic construct
  misc_feature 1..23: note = Primer
  source 1..23: mol_type = other DNA
gtgagtacattcattgtactgtg